SEQUENCE LISTING (21 sequences)

SEQ ID NO: 1 - protein, 6 aa, Escherichia coli
RRSRRV

SEQ ID NO: 2 - protein, 10 aa, Artificial Sequence
  Description of Artificial Sequence Synthetic peptide
KTTGGRIYGG

SEQ ID NO: 3 - protein, 7 aa, Artificial Sequence
  Description of Artificial Sequence Synthetic peptide
QARGGSK

SEQ ID NO: 4 - protein, 8 aa, Artificial Sequence
  Description of Artificial Sequence Synthetic peptide
GLQRALEI

SEQ ID NO: 5 - protein, 9 aa, Artificial Sequence
  Description of Artificial Sequence Synthetic peptide
KYLGRSYKV

SEQ ID NO: 6 - protein, 7 aa, Artificial Sequence
  Description of Artificial Sequence Synthetic peptide
IEFDSGK

SEQ ID NO: 7 - protein, 11 aa, Artificial Sequence
  Description of Artificial Sequence Synthetic peptide
  N term 5FAM
  MOD_RES (1)..(1): D-amino acid
  MOD_RES (8)..(8): Lys-CPQ2
  MOD_RES (9)..(9): D-amino acid
  MOD_RES (10)..(10): D-amino acid
  MOD_RES (11)..(11): D-amino acid
AIEFDSGKKKC

SEQ ID NO: 8 - protein, 4 aa, Artificial Sequence
  Description of Artificial Sequence Synthetic peptide
  N term 5TAMRA
  MOD_RES (4)..(4): Arg-QXL570
RTKR

SEQ ID NO: 9 - protein, 7 aa, Artificial Sequence
  Description of Artificial Sequence Synthetic peptide
  N term 5FAM
  MOD_RES (7)..(7): Gly-QXL520
ENLYFQG

SEQ ID NO: 10 - protein, 7 aa, Artificial Sequence
  Description of Artificial Sequence Synthetic peptide
RRSRRVK

SEQ ID NO: 11 - protein, 7 aa, Artificial Sequence
  Description of Artificial Sequence Synthetic peptide
  N term DABCYL
  MOD_RES (7)..(7): Lys-5FAM
RRSRRVK

SEQ ID NO: 12 - protein, 8 aa, Artificial Sequence
  Description of Artificial Sequence Synthetic polyarginine sequence
RRRRRRRR

SEQ ID NO: 13 - protein, 23 aa, Artificial Sequence
  Description of Artificial Sequence Synthetic peptide
EEEEEEEEEEERKTRRRRRRRRR

SEQ ID NO: 14 - protein, 24 aa, Artificial Sequence
  Description of Artificial Sequence Synthetic peptide
EEEEEEEEENLYFQGRRRRRRRRR

SEQ ID NO: 15 - protein, 8 aa, Artificial Sequence
  Description of Artificial Sequence Synthetic peptide
  N term 5FAM
  MOD_RES (7)..(7): Lys-CPQ2
LQRIYKKC

SEQ ID NO: 16 - protein, 12 aa, Artificial Sequence
  Description of Artificial Sequence Synthetic peptide
  N term 5FAM
  MOD_RES (11)..(11): Lys-CPQ2
KSVARTLLVKKC

SEQ ID NO: 17 - protein, 10 aa, Artificial Sequence
  Description of Artificial Sequence Synthetic peptide
  N term 5FAM
  MOD_RES (9)..(9): Lys-CPQ2
QRQRIIGGKC

SEQ ID NO: 18 - protein, 11 aa, Artificial Sequence
  Description of Artificial Sequence Synthetic peptide
  N term 5FAM
  MOD_RES (10)..(10): Lys-CPQ2
KYLGRSYKVKC

SEQ ID NO: 19 - protein, 11 aa, Artificial Sequence
  Description of Artificial Sequence Synthetic peptide
  N term 5FAM
  MOD_RES (9)..(9): Lys-CPQ2
  MOD_RES (10)..(10): D-amino acid
GLQRALEIKKC

SEQ ID NO: 20 - protein, 4 aa, Artificial Sequence
  Description of Artificial Sequence Synthetic peptide
RTKR

SEQ ID NO: 21 - protein, 7 aa, Artificial Sequence
  Description of Artificial Sequence Synthetic peptide
ENLYFQG

(19) United States
(12) Patent Application Publication
(10) Pub. No.: US 2022/0249369 A1
(43) Pub. Date: Aug. 11, 2022

(54) BIOLOGICAL BITS AS COMPUTABLE ELEMENTS WITHIN LIVING SYSTEMS

(71) Applicant: GEORGIA TECH RESEARCH CORPORATION, Atlanta, GA (US)

(72) Inventors: Brandon HOLT, Atlanta, GA (US); Gabriel KWONG, Atlanta, GA (US)

(73) Assignee: GEORGIA TECH RESEARCH CORPORATION, Atlanta, GA (US)

(21) Appl. No.: 17/276,711

(22) PCT Filed: Sep. 19, 2019
(86) PCT No.: PCT/US19/51833

§ 371 Date: Mar. 16, 2021

Related U.S. Application Data
(60) Provisional application No. 62/733,287, filed on Sep. 19, 2018.

(51) Int. Cl. A61K 9/00; G16B 5/20; G01N 33/68; G16B 40/30; A61K 47/62; A61K 47/69

(52) U.S. Cl. CPC A61K 9/0097; G16B 5/20; A61K 47/6911; G16B 40/30; A61K 47/62; G01N 33/6848

(57) ABSTRACT

Disclosed herein are protease-based biological circuits for use in the diagnosis and treatment of disease and disorders characterized by aberrant protease signaling. An exemplary method of treating disease in a subject includes administering to the subject a plurality of therapeutic agent loaded-liposomes, wherein each of the liposomes comprises a different density of peptides surrounding the core and a different dose of the therapeutic agent, wherein the peptides comprise a cleavage site for a protease of interest, and wherein cleavage of the peptides open the liposome and release the therapeutic agent from within the liposome.

### TECHNICAL FIELD OF THE INVENTION

This invention is generally related to the diagnosis and treatment of diseases and disorders characterized by aberrant protease signaling.

### BACKGROUND OF THE INVENTION

Rapid advances in engineered biological circuits are motivating the design of new treatment and detection platforms for practical applications in programmable medicine. The development of foundational components, such as molecular logic gates (Baron, et al., J Phys Chem, 110:8548-8553 (2006)) and genetic clocks (Elowitz, et al., Nature, 403:335 (2000); Tigges, et al., Nature, 457:309-312 (2009)) have enabled the design of biocircuits with increasing complexity, including the ability to solve mathematical problems (Adleman, L. M., Science, 266:1021-2014 (1994)), build autonomous robots (Amir, et al., Nature Nanotechnology, 9:353 (2014)), and play interactive games (Pei, et al., Nature Nanotechnology, 5:773-777 (2010)). Recently, programmable biocircuits have been applied for therapeutic and diagnostic applications (Higashikuni, et al., Current Opinion in Biotechnology, 47:133-141 (2017)), including genetic circuits that sense-and-respond to dysregulated inflammation (Smole, et al., Molecular Therapy, 25:102-119 (2017)) or blood glucose levels (Xie, et al., Science, 354:1296-1301 (2016)). To date, the design of these biocircuits is principally focused on constructs that are implemented in cell-based platforms—which require genome or protein engineering (Brophy, & Voigt, Nature Methods, 11:508 (2014); Karginov, et al., Nature Biotechnology, 28:743 (2010) Dagliyan, et al., PNAS, 110:6800-6804 (2013); Guntas, et al., PNAS, 102:11224-11229 (2005))—and carry out algorithms inspired by classical computer circuits, which operate on binary digits (bits) and Boolean logic gates (e.g., AND, OR, NAND).

By contrast, the ability to perform inference-based tasks—such as identification of which single input cause resulted in the observed output effect given multiple plausible inputs—are more challenging. In contrast to classical circuits, probabilistic circuits, which operate on analog bits characterized by a probability distribution of states, efficiently solve inference problems by assigning a likelihood probability that each plausible input would produce the observed output (Kevin, M., Machine Learning: A Probabilistic Perspective, The MIT Press (2012)). Probabilistic bits have been implemented with magnets (p-bits) (Camsari, et al., arXiv e-prints, (2018); Camsari, et al., Physical Review X, 7:031014 (2017); Zeeshan Pervaiz, et al., arXiv e-prints (2017)) as well as photons and electrons in quantum systems (qubits) (Ladd, et al., Nature, 464:45 (2010); Nielson & Chuang, Quantum Computational and Quantum Information, Cambridge University Press, 2018. In medicine, differential diagnoses are fundamentally based on inference, wherein an observed symptom could be caused by several diseases. Conversely, the decision to treat a patient is determined by a clear set of inputs (e.g., disease stage, biomarker level, etc.) (Higashimuni, et al., Current Opinion in Biotechnology, 47:133-141 (2017)).

The dysregulated activity of protease networks is a hallmark of complex diseases which has provided the impetus to use measurements of protease activity as biomarkers of disease and disease severity. Modern diagnostics and therapeutics have made significant headway with the development of genetic biomarkers, targeted therapy, and immune modulation to enable precision medicine for diseases such as cancer and autoimmune diseases. However, there are fundamental limitations to the detection of cancers and other diseases using blood biomarkers (the current standard for biomarker detection) due to non-specificity, dilution, and degradation of analytes. There is a need for more specific and sensitive methods for measuring protease biomarkers in diseases patients.

Therefore, it is an object of the invention to provide systems and methods for diagnosing and treating disease in subjects in need thereof.

It is another object of the invention to provide systems and methods for detecting and measuring protease activity in a subject.

### SUMMARY OF THE INVENTION

Disclosed herein are systems and methods for the detection and quantification of protease activity in a biological sample from a subject suspected of having a disease characterized with aberrant protease signaling. The systems and methods can additionally be used to deliver regulated amounts of therapeutics to the subject to treat disease. In such embodiments, the proteases within the subject serve to both release and titrate drug delivery within the subject.

One embodiment provides a drug delivery circuit including a plurality of biocomparators, and a plurality of activatable drug delivery agents, wherein the plurality of biocomparators perform a round of computation to determine if conditions are present to activate the drug, and if so then activate the appropriate drug delivery agent to deliver a drug to a subject. The biocomparators can have a biologically inert structure having a core and being surrounded by a density of cleavable peptides. In one embodiment, the biocomparator are a liposome. The activatable drug delivery agents can be activated by cleavage of a peptide surrounding the agent, by means such as protease cleavage or chemical cleavage. The biocomparator core can contain agents to activate the drug delivery agent. It can additionally contain proteases and protease inhibitors, wherein the proteases activate the drug delivery agents and the inhibitors inhibit the proteases such that the unique combination of protease and inhibitor cleaves only a specific drug delivery agent. The disclosed biocomparators can sense the amount of diseased cells or infectious agent in a sample or subject. This detected amount of diseased cells or infectious agent in a sample or subject cause one of the plurality of biocomparators to open and release the contents of the core, wherein the contents of the core cleave a specific drug delivery agent. The plurality of activatable drug delivery agents include different doses of a therapeutic agent. The amount of protease present in the subject cleaves the peptides of specific densities and releases the minimum amount of therapeutic agent necessary to treat the disease. The biocomparators and drug delivery agents that are not opened get filtered and excreted from the subject without releasing their therapeutic agent contents.

An exemplary method for treating disease in a subject in need thereof includes, contacting a biological sample from the subject with a plurality of biocomparators, wherein each biocomparator is surrounded by peptides that can be cleaved by a protease, each of the plurality of biocomparators have a different density of peptide surrounding it, and each of the plurality of biocomparators have a unique detectable molecule within its core; detecting the molecules released from each of the plurality of biocomparators; assigning a binary digit to the sample, wherein each detectable molecule is pre-assigned a binary digit that corresponds to a threshold protease level, and the presence of each detectable molecule or combination of detectable molecules present in the sample determines the binary digit; diagnosing the subject with disease if the binary digit indicates the protease level is above the threshold level; and administering to the subject the minimum effective dose of a therapeutic agent necessary to treat the disease or disorder.

In one embodiment, the detectable molecule within the biocomparator is a fluorescent molecule, a bioluminescent molecule, a mass-tag, or a protease substrate flanked by a quencher molecule and a fluorescent molecule. In another embodiment, the biocomparator optionally further includes signal proteases and protease inhibitors. Detecting the molecules can include subjecting the sample to mass spectrometry, flow cytometry, or ELISA. In some embodiment, the biological sample is a urine sample or a blood sample.

The subject can have cancer or a bacterial or viral infection. In some embodiment, the subject is administer a therapeutic agent such as an immunotherapeutic agent or an immunosuppressive agent. In other embodiments, the subject is administered an antimicrobial or antiviral agent.

In some embodiments, the peptides surrounding the biocomparator can be cleaved by a cancer-associated protease such as but not limited to matrix metalloproteinases, kallikreins, ADAM10, ADAM 17, cathepsin B, cathepsin L, cathepsin S, uPA, uPAR, PSA, and caspases-3, -6, -7, and -8. In other embodiments, the peptides can be cleaved by bacterial peptides such as OmpT.

### BRIEF DESCRIPTION OF THE DRAWINGS

FIG. 1A is a schematic illustration of using biological bit for programmable medicine. The binary state of a classical bit represented as two orthogonal states (0 or 1). A classical biological bit exists in a state of either high or low protease activity, defined by an activity threshold. The binary state of probabilistic bits represented as a superposition of state 0 or state 1. A probabilistic biological bit acting on two state substrates has two cleavage velocities (v₀ and v₁), which are the probabilities of observing the bbit in either state 0 or state 1. FIG. 1B is an overview schematic showing that the binary biological bits were used to construct a therapeutic digital drug dosing circuit to selectively lyse bacteria. FIG. 1C is a schematic showing the use of multi-state (m) probabilistic bbits to diagnose tissue factor-induced thrombosis in a mouse model.

FIG. 2A is a biocircuit diagram depicting the conversion of a biological input (protease activity) into a digital output with biocomparators, priority encoding, and OR gates. Circular arrow around grey protease represents enzyme activity. FIGS. 2B and 2C are graphs showing protease activity of bare (FIG. 2B) or peptide-caged (FIG. 2C) liposomes open by lipase or GzmB activity, respectively, to release TEV protease that cleaves a quenched peptide substrate. FIG. 2D is a protease orthogonality map measuring GzmB, WNV, and TEV protease activity against respective substrates alone and in the presence of WNV protease inhibitor. FIG. 2E is a bar graph showing cleavage velocity of increasing concentrations of GzmB across four orders of magnitude. FIG. 2F is a graph showing digital output as a function of input GzmB concentration.

FIG. 3A is a circuit diagram of a flash ADC. FIG. 3B is a truth table in which an input which activates a biocomparator produces a value of 1. To give this input priority, all biocompartators below are turned off, signified by value of x. Digital output bits are shaded and correspond to the 2-bit output of the ADC. FIG. 2C is a logic circuit diagram for one example input/output case through a 4-2 bit ADC. Input signal >V2 turns on d₀-d₂, but priority is given to d₂, which only turns on bit q₁, producing the output 10.

FIG. 4A is a graphic of cholesterol-anchored poly(acrylic acid) (CPAA) embedded in liposome membrane, crosslinked by amine terminated peptides. Carboxylic acid side groups on poly(acrylic acid) are activated by EDC*Mel. N-terminal amine and C-terminal amine (from lysine side chain) act as primary amines to react with activated CPAA side chains. FIG. 4B is a graph showing DLS size measurement of liposomes before and after peptide cage construction. FIG. 4C is a heat map showing concentration of GzmB required to unlock each level. Signal is measured via released protease cutting substrate, normalized to the negative control.

FIG. 5A is a graph showing phospholipase C-triggered release of FITC contained in liposomes. The negative control contains liposome only and no liposome. FIG. 2B is a graph showing phospholipase C and signal protease GzmB triggered release of FITC. Negative control contains lipase, but no signal protease.

FIGS. 6A-6D are schematics of the four possible signal conversions in the biological four-two bit analog-to-digital converter. The signal protease cleaves the peptide cage surrounding the biocomparators. Higher activity levels of GzmB result in more biocomparator levels being unlocked. Lipase is co-incubated with the bioADC such that all exposed biocomparators are fully opened via degradation of liposome by phospholipase C. Released signal converter proteases and priority encoding inhibitors interact to produce one digital signal. This signal interacts with OR gates to produce “high” or 1 values for the correct binary digit. FIGS. 6E-6R are graphs showing kinetic fluorescent data from 4-to-2 bit biological ADC, which is plotted in FIG. 2E.

FIG. 7A is a schematic of a biocircuit depicting the use of an ACD to quantify bacteria and autonomously unlock digital drug doses. Circular arrow represents enzyme activity. FIG. 7B is a graph showing the results of a cleavage assay measuring recombinant OmpT and live E. coli culture cleavage of peptide RRSRRV (SEQ ID NO:1) (n=3, two-way ANOVA and Sidak's multiple comparisons). FIG. 7C is a graph showing EC50 measurement for drug cytotoxicity and hemolysis against E. coli and red blood cells, respectively. Grey shading represents therapeutic window with 100% cytotoxicity and no hemolysis. FIG. 7D is a graph showing the viability of bacteria after treatment with locked drug and locked drug+protease. (n=4, one-way ANOVA & Dunnett's multiple comparisons to bacteria only control; scale bar=4 mm). FIGS. 7E-7H are graphs showing drug bacteria cytotoxicity and red blood cell hemolysis at five concentrations of bacteria with 4 different versions of the antimicrobial program each containing a different number of biocomparators (two-way ANOVA & Sidak's multiple comparisons; hemolysis n=2, cytotoxicity n=3). The four biocomparators, b₀, b₁, b₂, and b₃, were cross-linked at peptide cage reaction ratios, 0.0, 3.4×10⁻², 3.4, and 340 μmol/g (peptide:liposome), respectively. Line shading and error bars are standard deviation. RFU stands for “Relative Fluorescence Unit” and is plotted as fold change (FC) from initial fluorescence at time=0. n.s.=not significant, *<0.05, **<0.01, ***<0.001, and ****<0.0001.

FIGS. 8A-8B show the results of unlocking peptide caged liposomes with increasing peptide crosslinking densities (levels 0-8). Eight levels of increasing peptide cage crosslinking were used to determine the number of bacteria required to unlock each level. FIG. 8C is a graph showing bacterial cytotoxicity measurement of drug-loaded liposomes. Conditions are moving from left to right: Bacteria only control, bacteria plus free drug, bacteria plus drug-loaded liposome without lipase, and bacteria plus drug-loaded liposome with lipase. Samples were incubated with bacteria at 37° C. for eight hours and plated. CFU were quantified to estimate bacteria viability. Error bars are plotted as standard deviation (n=3). RFU stands for “Relative Fluorescence Unit” and is plotted as fold change (FC) from initial fluorescence at time=0.

FIG. 9 is a schematic illustration showing all possible drug doses from bioprogram. The OR gate 0 linked to bit p₀ outputs ⅓ of the available antimicrobial peptide (AMP) dose, whereas the OR gate 1 linked to bit p₁ outputs ⅔ of the available AMP dose. This translates each digital output to a drug dose increasing by units of ⅓ the total dose.

FIG. 10A is a schematic illustration showing Michaelis-Menten model of protease-substrate reactions in a bulk mixture. FIG. 10B is a graph showing a protease cleavage velocity plot of thrombin cleaving two different peptide substrates (KTTGGRIYGG (SEQ ID NO:2) and QARGGSK (SEQ ID NO:3)). FIG. 10C is a schematic depicting the major steps and probabilities in the single protease Gillespie algorithm. FIG. 10D is graph showing a simulated cleavage plot of a single protease against two different peptide substrates with different. FIG. 10E is a graph showing a simulation of the cumulative kinetic activity of 105 individual proteases. FIG. 10F is a schematic depicting simulations of 625 unique conditions by varying relative concentrations and complexation probabilities for either substrate. The total number of substrates cut, and the initial velocity was calculated for each case, and each were used to estimate the probability of the protease cleaving substrate-0. FIG. 10G is a set of graphs showing simulation for a single unique case comparing the probability of a protease cleaving substrate-0 or substrate-1 based on substrate counts (i.e. p⁰_{counts} or p¹_{counts} or velocity (i.e. p⁰ᵥₑₗ or p¹ᵥₑₗ) over time. FIG. 10H is a scatter plot representing the correlation between the probability of protease existing in state 0 as estimated by relative cleavage velocities (i.e., p⁰ᵥₑₗ) vs. probability as estimated by counting individual Bayesian events (p⁰_{counts}).

FIG. 11A is a schematic detailing an example Oracle problem. FIG. 11B is a schematic demonstrating on-target, multi-target, and common-target protease activity. FIG. 11C is a schematic showing the workflow involved in solving the one example of the two-bit oracle problem. FIG. 11D is a schematic of calculating the probabilities of all 8 possible outputs from the 2-bit oracle problem by multiplying relative cleavage velocities (vn) of each bbit.

FIGS. 12A-12C are truth tables depicting ideal inputs and outputs of a Uniform (U) gate. FIGS. 12D-12F are graphs showing single protease Gillespie-algorithm simulations demonstrating the function of a U-gate to create an equal superposition of state 0 and 1. A second consecutive U-gate operation reverts bbit back to original state. FIGS. 12G-12I are graphs showing the implementation of the biological U gate on a two-state probabilistic bit. A protease (plasmin), first only exposed to the state 0 substrate (GLQRALEI (SEQ ID NO:4)), is then exposed to the state 1 substrate (KYLGRSYKV (SEQ ID NO:5)), resulting in similar probabilities of being observed in either state (˜45% and 55% respectively, n=3). A second U-gate operation, performed by adding state 0 substrate in large molar excess, reverses the operation to restore b0 to its original state 0 probability (˜93%, n=3). FIGS. 12J-12M are truth tables depicting the ideal inputs and outputs of the Linker (L) gate. If the control bit (b₀) has a non-zero probability of occupying state 1, then the control bit determines the output of the target bit (b₁) to a user defined value, 1−y′. FIGS. 12N-12Q are graphs showing the experimental operation of the L-gate. The output states of control protease bit (plasmin) and target protease bit (thrombin) are matched by addition of the state 1 substrate to thrombin such that the output probabilities for both bbits are matched. FIG. 12R is a schematic showing how biological scores represent all four possible configurations (00, 01, 10, 11) of the 2-bit oracle problem. FIG. 12S is a graph showing how biological bits solve all four implementations of the two-bit oracle problem; b₀, b₁, and b₂ are bits with two possible states (i.e., 0 and 1) with associated probability ranging from 0.0-1.0. The individual entries on the x-axis describe which state b₀, b₁, or b₂ are occupying for that particular output. rcv=relative cleavage velocities.

FIGS. 13A-13C are graphs showing single protease simulation (Gillespie algorithm), of two proteases: control protease (FIG. 13A, black dot) and target protease (FIGS. 13B-13CB, circle). Control bit probability p(1) controls the p(1) of target bit. FIGS. 13D-13G are graphs showing experimental operation of the L-gate. The output states of control protease bit (plasmin) and target protease bit (thrombin) are matched by addition of the state 1 substrate to thrombin such that the output probabilities for both bbits are matched at 1.0. FIGS. 13H-13K are graphs showing the results of simulating the three bit oracle problem with three single protease simulations. In each case, 0 (i.e., 00), 1 (i.e., 01 and 10), or 2 (i.e., 11) proteases have similar activity to the output bit (b₂).

FIG. 14A is a schematic illustration showing a coagulation cascade as a prototypic protease network in vivo. The cascade is activated by two major pathways (i.e., intrinsic or extrinsic). Tissue factor acts as a transmembrane receptor for Factor VIIa. Activation of the coagulation cascade ultimately results in the formation of fibrin clots catalyzed by the protease thrombin. FIG. 14B is a schematic illustration of protease bit sampling assay (PBSA). (1) The relative cleavage activity of a protease network is quantified using protease substrates (i.e., peptides). Multiple proteases, n, (e.g., plasmin, factor Xa, factor IXa, thrombin, etc.) simultaneously cleave a common-target substrate. (2) Multiple different common-target substrate sequences, m, are used to sample different subsets of proteases and the relative cleavage velocity (rcv) of each substrate is quantified. (3) To estimate the state probabilities (i.e., Prob.) of a probabilistic protease bit, the relative cleavage velocities (i.e., rcv) are compared. (4) The variance of state probabilities is then used to classify disease organisms form healthy organisms. FIG. 14C is a schematic showing computational simulations of AUROCs (area under the receiver operating characteristics) by PBSA as a function of the number of m-state substrates and number of dysregulated proteases. Protease dysregulation was simulated by varying 0, 20, 100, or 550 proteases by scaling their activity by a factor of five (upregulation) or one-fifth (downregulation), respectively. To simulate promiscuous sampling, a substrate library of size m (ranging from 2-550) was modeled, where each member of the library randomly sampled n proteases (ranging from 1-550). The total number of substrates cut was calculated by summing the activity scores for each of the n sampled proteases. The variance of the measured substrates cleaved was used to classify healthy from disease organisms. Results from each classification are plotted as AUROCs (i.e., area-under-the-receiver-operator characteristic curve, y-axis) vs. number of state substrates, m (x-axis).

FIG. 15A-15C are graphs showing the results of cleavage assays measuring complement (C1r, FIG. 15A) and coagulation (thrombin, FIG. 15B; plasmin, FIG. 15C) protease specificity towards Substrates 1-7, respectively. Results reported as relative cleavage velocity (rcv). FIG. 15D is a graph showing results of a human serum complement activation assay to measure specificity of proteases in the classical complement cascade towards Substrates 1-7. RFU stands for “Relative Fluorescence Unit” and is plotted as fold change (FC) from initial fluorescence at time=0.

FIG. 16A is a schematic illustration showing in vitro experiments measuring the variance in bit states of complement (e.g., C1r, MASP2, factor D, factor I) and coagulation (e.g., factor XIIa, factor Xa, plasmin, thrombin, protein C) proteases with seven state substrates (numbered 1-7). FIGS. 16B-16C are graphs showing probability distribution of seven cleaved substrates (1-7) for in vitro cocktail of recombinant complement proteases (FIG. 16A) or coagulation proteases (FIG. 16B) are used to estimate state probabilities of a 7-state protease bits. Results are plotted as relative cleavage velocity. Error bars represent standard deviation (n=5). FIG. 16D is a graph showing the results of using variance of substrates cleaved sampled from 7-state protease bits (i.e., PBSA) to classify the protein cocktails as complement or coagulation. Results plotted as receiver operating characteristic (ROC) and classification accuracy is quantified as area under the ROC (i.e., AUROC). FIG. 16E is a schematic illustration showing the activity sensor workflow. (1) Protease substrates (i.e., activity sensors) were injected (i.e., tail vein injection) into mice. (2) Nanoparticles carrying substrates are cleaved by coagulation proteases as they form fibrin clots. (3) Cleavage products and nanoparticles pass through the kidneys, which filter out uncleaved nanoparticle+substrate and filter cleaved peptides into urine. (4) Urine is collected and the quantity of each mass tag reporter is measured via mass spectrometry. FIGS. 16F-16G are graphs showing results of urinalysis measuring the concentration of each of the seven bit-states (i.e., substrates) on Day 0 (healthy, FIG. 16F) and Day 4 (disease, FIG. 16G) plotted as mean, where error bars are standard deviation (n=7). FIG. 16H is a graph classifying disease mice using the D-dimer ELISA. FIG. 16I is a graph classifying disease mice by sampling probabilistic protease bits in a murine model of pulmonary embolism.

FIG. 17A is a graph showing quantification of near-infrared images of lungs from vehicle (fibrinogen-VT750 only) and experimental (fibrinogen-VT750 and tissue factor) mice in arbitrary fluorescence units. Means were compared with unpaired, two-tailed t-test. FIG. 17B is a graph showing results of a murine d-dimer ELISA quantifying concentration of d-dimer in plasma collected from vehicle (fibrinogen-VT750 only) and experimental (fibrinogen-VT750 and tissue factor) mice 30 minutes and 130 minutes post-injection with tissue factor. Means were compared by one-way ANOVA followed by Dunnett's multiple comparisons test. Error bars represent standard deviation. n. s.=not significant, *<0.05, **<0.01, ***<0.001, and ****<0.0001.

FIG. 18A is a schematic of mass-barcoded protease activity sensor. Iron oxide nanoparticles (i.e., carrier) are conjugated to peptide substrates which are uniquely labeled with mass barcodes (i.e., reporter). FIG. 18B is a graph showing Dynamic Light Scattering (DLS) measurement of nanoparticle size distribution. FIG. 18C is a graph showing absorbance spectra for iron oxide nanoparticles (IONP) only and IONP conjugated to a substrate, measured in 5 nm steps from 400 to 800 nm.

FIGS. 19A-19D and 19I-19K are graphs showing relative substrate probabilities measured via urinalysis of the seven mice before and after onset of pulmonary embolism (Injection 1). FIGS. 19E-19H and 19L-19N are graphs showing extraction of protease activity probability distribution profiles before and after onset of pulmonary embolism. “rcv” stands for relative cleavage velocity.

FIG. 20 is a graph showing the effect of the number of substrates on classification accuracy.

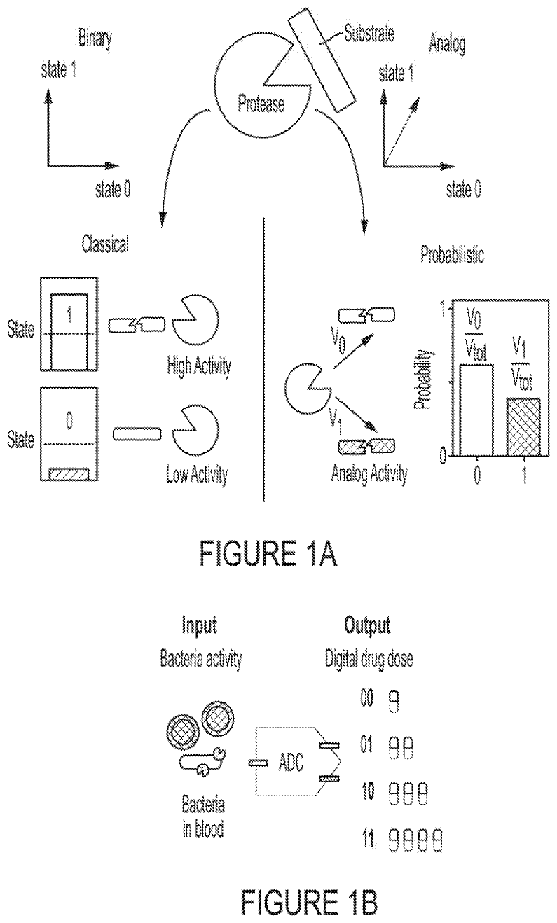

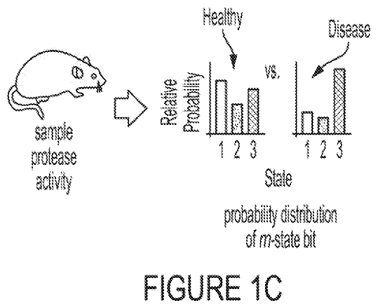

### DETAILED DESCRIPTION OF THE INVENTION

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, a “protease” is an enzyme that catalyzes proteolysis, the breakdown of proteins into smaller polypeptides or single amino acids. A “protease substrate” is a protein that is cleaved by a protease. Protease substrates contain cleavage sites that are recognized by the protease.

As used herein, a “biological circuit” is an application of synthetic biology where biological parts inside a cell or biological system are designed to perform logical functions mimicking those observed in electronic circuits.

As used herein, a “digital output” refers to an output that can be represented in a binary format. The binary numeral system, or base-2 number system represents numeric values using two symbols, 0 and 1. More specifically, the usual base-2 system is a positional notation with a radix of 2. Owing to its implementation in digital electronic circuitry using logic gates, the binary system is used internally by all modern computers. A “bit,” as defined herein, is a binary digit.

As used herein, a “biocomparator” is a compound that detects an input biological signal and compares it to a stored reference signal. If the input signal is greater than the reference signal then the biocomparator produces output A (e.g., binary output=1), and if the input signal is less than the reference signal, then the biocomparator produces output B (e.g., binary output=0).

### II. Protease Based Systems and Methods for Diagnosing and Treating Disease

Disclosed herein are protease-based biological circuits and methods of use thereof to diagnose and treat diseases and disorders. Exemplary biological circuits disclosed herein include analog-to-digital converters and logic gates.

### A. Drug Delivery Circuit

Disclosed herein is a drug delivery circuit to convert continuous biological signals into detectable signals or binary digits. A central function of complex circuits is the ability to store and manipulate digitized information. An electronic ADC performs three major operations during signal conversion: voltage comparison, priority assignment, and digital encoding. An analog voltage is first compared against a set of increasing reference voltages (V₀-Vᵢ) by individual comparators (d₀-dᵢ) that allow current to pass if the input signal is greater than or equal to the reference value. During priority assignment, only the activated comparator with the highest reference voltage, dₙ, remains on while all other activated comparators, dₙ₋₁-d₀ are turned off. The prioritized signal is then fed into a digital encoder comprising OR gates to produce binary values.

In one embodiment, the disclosed ADC biocircuit uses proteases as the core signal instead of voltage as is typical of classical electronic ADCs. Proteases are a class of enzymes that includes over 550 members encoded within the human genome. Proteases catalyze proteolysis, the breakdown of proteins into smaller polypeptides or single amino acids. They do this by cleaving the peptide bonds within proteins by hydrolysis. Because of their ability to alter proteins, proteases are involved in regulating the fate, localization, and activity of proteins, modulating protein-protein interactions, creating new bioactive molecules, contributing to the processing of cellular information, and generating, transducing, and amplifying molecular signals. Proteases can influence DNA replication and transcription, cell proliferation and differentiation, tissue morphogenesis and remodeling, heat shock and unfolded protein responses, angiogenesis, neurogenesis, ovulation, fertilization, wound repair, stem cell mobilization, hemostasis, blood coagulation, inflammation, immunity, autophagy, senescence, necrosis, and apoptosis.

The disclosed ADC biocircuit uses biocomparators surrounded with specific peptides (peptide caged biocomparators) to determine protease presence and activity in a sample, and further diagnose or treat disease. In one embodiment, the peptide caged biocomparators are surrounded by an increasing density of peptides, which have the ability to be cleaved by a protease of interest. In such an embodiment, the cleavage of the peptides surrounding the biocomparator core opens the biocomparator releasing the contents, if any. A library of peptide caged biocomparators can be prepared, each one containing unique cargo that can be used to identify it. In one embodiment, the peptide caged biocomparators contain inhibitors and signal proteases that can activate additional signal molecules such as fluorescent reporters to generate a readable output signal. The most highly activated peptide caged biocomparators indicates the protease presence and activity level in the input sample. Further details of each component of the system and method are described below.

### 1. Peptide Caged Biocomparators

In the disclosed biological ADC circuit, the input protease signal (the biological sample being analyzed) is compared to a reference protease signal such that the input protease is assigned binary state 1 only if it exceeds a threshold level of activity. The reference signal is calculated using biological analogs of comparators. In one embodiment, the biological analogs of comparators are engineered biocomparators locked by an outer peptide cage. In such an embodiment, the biocomparators serve to reference the level of input protease activity required to fully degrade the peptide cage and expose the lipid core, analogous to reference voltages stored in electronic comparators. In one embodiment, the biocomparator is a liposome.

In one embodiment, the peptide that surrounds the biocomparator is a protease substrate. Protease substrates are proteins that contain a recognition sequence for a protease to cleave. In some embodiments, the proteases and substrates are involved in diseases or infections. Exemplary proteases involved in cancer include but are not limited to matrix metalloproteinases such as MMP1, MMP2, MMP3, MMP8, MMP9, MMP12, MMP13, and MMP14, kallikreins such as KLK1, KLK2, KLK3, KLK6, and KLK7, ADAM10, ADAM 17, cathepsin B, cathepsin L, cathepsin S, uPA, uPAR, PSA, cysteine proteinases of the caspase family, such as caspase-3, -6, -7, -8. Proteases involved with other diseases include but are not limited to cathepsin G, neutrophil elastase, proteinase 3, mucosa-associated lymphoid tissue 1 (MALT1), granzymes, pappalysins, neprilysin, angiotensin-converting enzyme, metallocarboxypeptidases, glutamate carboxypeptidase II, elastin, coagulation factors such as thrombin, factor VIIa, factor IXa, and factor Xa, tissue-type plasminogen activator, cathepsin D, cathepsin E, and cathepsin K. In another embodiment, the proteases are viral proteases such as but not limited to HIV protease, TEV protease, Herpesvirus protease, adenovirus protease, and hepatitis C virus protease.

In other embodiments, the peptide is a substrate for a bacterial protease. In some embodiment, the protease is a bacterial surface expressed protease. Exemplary bacterial proteases include but are not limited to E. coli proteases such as OmpT, OmpP, ElaD, heat shock protein 31, putative Cys protease YhbO, DegP, DegQ, and DegS, Yersinia pestis Pla, Salmonella enterica PgtE, C. difficile A and B toxin proteases, B. anthrasis lethal factor protease and Shigella flexneri SopA.

In one embodiment, a plurality of caged liposomes are prepared with varying density of peptide surrounding the liposome core. The peptide to liposome ratio can be from 0 to about 500 μmol/g. In some embodiments, the peptide to liposome ratio is 0, 1×10⁻⁵, 1×10⁻⁴, 1×10⁻³, 0.01, 0.1, 0.5, 1, 5, 10, 15, 20, 25, 50, 75, 100, 125, 150, 175, 200, 225, 250, 275, 300, 325, 350, 375, 400, 425, 450, 475, or 500 μmol/g. The level of protease activity necessary to degrade the peptide cage and expose the liposome core is calculated for each reference liposome to be used in a biological ADC circuit.

### a. Cargo

The peptide caged biocomparators can include other molecules within their core that are used to further convert the input protease signal into a detectable signal or digital output. These molecules can be used to perform the equivalent of priority assignment and gating, ensuring that the highest activated liposome is detected.

### i. Signal Molecules

In one embodiment, the core of the peptide caged biocomparator further includes detectable signal molecules that can be measured to determine protease levels within a sample or a subject. In such an embodiment, the caged biocomparators contain a different detectable signal for each peptide density. For example, a liposome having a peptide to liposome ratio of 1×10⁻⁵ could contain a GFP-reporter and a liposome having a peptide to liposome ratio of 0.1 could contain an mCherry reporter. The amount of protease required to fully degrade the peptide surrounding each liposome can be recorded based on the presence of fluorescent signal. Other detectable signal molecules that could be included in the core of the liposome include but are not limited to avidin, biotin, beta-galactosidase, luciferase, alkaline phosphatase (AP), and horseradish peroxidase (HRP).

In some embodiments, the detection of a signal molecule or combination of signal molecules is correlated to a specific digital output signal. In such an embodiment, each unique signal molecule is linked to a peptide caged biocomparator with a known protease activity needed to open each liposome. Protease activity above a threshold level is assigned to binary state 1, whereas protease activity below a threshold level is assigned to binary state 0. The peptide caged liposome with the highest protease activity required to open it, is therefore assigned 1,1.

### ii. Inhibitors and Signal Proteases

In another embodiment, the biocomparators contain a unique combination of inhibitors and signal proteases. In one embodiment this provides a means of performing priority assignment on the activated biocomparators, such that the highest activated biocomparator is prioritized above other activated biocomparators. In such an embodiment, each different biocomparator class (i.e. different peptide density) is filled with a different, unique combination of inhibitors and/or signal peptides such that the detection of a specific signal peptide is associated with only one class of biocomparator. Each peptide caged biocomparator class has a pre-determined cleavage velocity associated with it such that the detection of the signal peptides from a specific biocomparator within the sample or subject indicates an amount of protease present in that sample or subject. The cleavage velocities can be experimentally determined using common methods known in the art such as protease cleavage assays.

In one embodiment, the biocomparators can contain any signal protease capable of cleaving a substrate. The signal protease within the biocomparator must be a different protease than the input protease being detected. Exemplary proteases can be bacterial, viral, murine, or human.

The biocomparators can also include protease inhibitors within their core. In some embodiments, the inhibitors should inhibit at least one of the signal proteases that are also contained within the biocomparator.

In some embodiments, the signal proteases released from the peptide caged biocomparators serve to produce further detectable signal to convert the input protease signal into a detectable signal or digital output. The signal proteases released from the peptide caged biocomparators can be used to cleave a peptide such that a quencher is cleaved from a fluorescent signal. In one embodiment, the peptide includes a quencher molecule and a fluorescent molecule flanking the protease cleavage site. Quencher molecules are known in the art. Exemplary quencher molecules include but are not limited to Deep Dark Quenchers (Eurogentec), DABCYL, TAMRA, BHQ-1®, BHQ-2®, BHQ-3®, BBQ®-650, ECLIPSE, Iowa Black® quenchers, and QSY. Exemplary fluorophores or fluorescent reporters include but are not limited to 6-FAM™, TET™, JOE™, HEX™, VIC®, cyanine 3, ROX™, LC Red 640, cyanine 5, fluorescein isothiocyanate (FITC), rhodamine (tetramethyl rhodamine isothiocyanate, TRITC, Oregon Green, Pacific Blue, Pacific Green, Pacific Orange, Texas Red, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, and Alexa Fluor 750.

In some embodiments, the presence of different fluorescent probes and/or combinations of the probes are translated to a digital output, for example a binary output. In one embodiment, the system is a 2-bit binary system wherein one fluorescent probe is output as 0, 1, the second fluorescent probe is output as 1, 0, and the presence of both is output as 1, 1.

### 2. Activatable Drug Delivery Agent

The disclosed drug delivery circuit also includes activatable drug delivery agents. In one embodiment, the activatable drug delivery agents can be liposomes containing a core of therapeutic agent wherein the agent is released only if the drug delivery agent is activated by an external signal. In other embodiments, the activatable drug delivery agent is a prodrug containing a cleavable linker, wherein the drug is not active until it is cleaved from the other moiety.

In some embodiments, the disclosed activatable drug delivery agents can include therapeutic agents within their core that are released in the presence of specific activation signals released from the biocomparator. In one embodiment, the drug delivery agents include different doses of therapeutic agent within the core. In such an embodiment, the amount or type of protease released from the peptide caged biocomparator is correlated to a specific amount of therapeutic agent required to treat the disease or disorder caused by the protease.

Therapeutic agents that are contemplated to be contained within the drug delivery agents are provided in detail below.

### a. PD-1 Inhibitors

Programmed Death-1 (PD-1) is a member of the CD28 family of receptors that delivers a negative immune response when induced on T cells. Contact between PD-1 and one of its ligands (B7-H1 or B7-DC) induces an inhibitory response that decreases T cell multiplication and/or the strength and/or duration of a T cell response. Suitable PD-1 antagonists are described in U.S. Pat. Nos. 8,114,845, 8,609,089, and 8,709,416, which are specifically incorporated by reference herein in their entities, and include compounds or agents that either bind to and block a ligand of PD-1 to interfere with or inhibit the binding of the ligand to the PD-1 receptor, or bind directly to and block the PD-1 receptor without inducing inhibitory signal transduction through the PD-1 receptor.

In some embodiments, the PD-1 receptor antagonist binds directly to the PD-1 receptor without triggering inhibitory signal transduction and also binds to a ligand of the PD-1 receptor to reduce or inhibit the ligand from triggering signal transduction through the PD-1 receptor. By reducing the number and/or amount of ligands that bind to PD-1 receptor and trigger the transduction of an inhibitory signal, fewer cells are attenuated by the negative signal delivered by PD-1 signal transduction and a more robust immune response can be achieved.

It is believed that PD-1 signaling is driven by binding to a PD-1 ligand (such as B7-H1 or B7-DC) in close proximity to a peptide antigen presented by major histocompatibility complex (MHC) (see, for example, Freeman, Proc. Natl. Acad. Sci. U.S.A, 105:10275-10276 (2008)). Therefore, proteins, antibodies or small molecules that prevent co-ligation of PD-1 and TCR on the T cell membrane are also useful PD-1 antagonists.

In some embodiments, the PD-1 receptor antagonists are small molecule antagonists or antibodies that reduce or interfere with PD-1 receptor signal transduction by binding to ligands of PD-1 or to PD-1 itself, especially where co-ligation of PD-1 with TCR does not follow such binding, thereby not triggering inhibitory signal transduction through the PD-1 receptor.

Other PD-1 antagonists contemplated by the methods of this invention include antibodies that bind to PD-1 or ligands of PD-1, and other antibodies.

Suitable anti-PD-1 antibodies include, but are not limited to, those described in the following U.S. Pat. Nos. 7,332,582, 7,488,802, 7,521,051, 7,524,498, 7,563,869, 7,981,416, 8,088,905, 8,287,856, 8,580,247, 8,728,474, 8,779,105, 9,067,999, 9,073,994, 9,084,776, 9,205,148, 9,358,289, 9,387,247, 9,492,539, 9,492,540, all of which are incorporated by reference in their entireties.

Exemplary anti-B7-H1 (also referred to as anti-PD-L1) antibodies include, but are not limited to, those described in the following U.S. Pat Nos. 8,383,796, 9,102,725, 9,273,135, 9,393,301, and 9,580,507 all of which are specifically incorporated by reference herein in their entirety.

For anti-B7-DC (also referred to as anti-PD-L2) antibodies see U.S Pat. Nos. 7,411,051, 7,052,694, 7,390,888, 8,188,238, and 9,255,147 all of which are specifically incorporated by reference herein in their entirety.

Other exemplary PD-1 receptor antagonists include, but are not limited to B7-DC polypeptides, including homologs and variants of these, as well as active fragments of any of the foregoing, and fusion proteins that incorporate any of these. In some embodiments, the fusion protein includes the soluble portion of B7-DC coupled to the Fc portion of an antibody, such as human IgG, and does not incorporate all or part of the transmembrane portion of human B7-DC.

The PD-1 antagonist can also be a fragment of a mammalian B7-H1, for example from mouse or primate, such as a human, wherein the fragment binds to and blocks PD-1 but does not result in inhibitory signal transduction through PD-1. The fragments can also be part of a fusion protein, for example an Ig fusion protein.

Other useful polypeptides PD-1 antagonists include those that bind to the ligands of the PD-1 receptor. These include the PD-1 receptor protein, or soluble fragments thereof, which can bind to the PD-1 ligands, such as B7-H1 or B7-DC, and prevent binding to the endogenous PD-1 receptor, thereby preventing inhibitory signal transduction. B7-H1 has also been shown to bind the protein B7.1 (Butte et al., Immunity, Vol. 27, pp. 111-122, (2007)). Such fragments also include the soluble ECD portion of the PD-1 protein that includes mutations, such as the A99L mutation, that increases binding to the natural ligands (Molnar et al., PNAS, 105:10483-10488 (2008)). B7-1 or soluble fragments thereof, which can bind to the B7-H1 ligand and prevent binding to the endogenous PD-1 receptor, thereby preventing inhibitory signal transduction, are also useful.

PD-1 and B7-H1 anti-sense nucleic acids, both DNA and RNA, as well as siRNA molecules can also be PD-1 antagonists. Such anti-sense molecules prevent expression of PD-1 on T cells as well as production of T cell ligands, such as B7-H1, PD-L1 and/or PD-L2. For example, siRNA (for example, of about 21 nucleotides in length, which is specific for the gene encoding PD-1, or encoding a PD-1 ligand, and which oligonucleotides can be readily purchased commercially) complexed with carriers, such as polyethyleneimine (see Cubillos-Ruiz et al., J. Clin. Invest. 119(8): 2231-2244 (2009), are readily taken up by cells that express PD-1 as well as ligands of PD-1 and reduce expression of these receptors and ligands to achieve a decrease in inhibitory signal transduction in T cells, thereby activating T cells.

### b. CTLA4

Cytotoxic T-lymphocyte-associated protein 4 (CTLA4) is a is a protein receptor that functions as an immune checkpoint and downregulates immune responses. CTLA4 is constitutively expressed in regulatory T cells but only upregulated in conventional T cells after activation. CTLA4 transmits an inhibitory signal to T cells. In some embodiments, the immunotherapeutic agent is an antagonist of CTLA4, for example an antagonistic anti-CTLA4 antibody. An example of an anti-CTLA4 antibody contemplated for use in the methods of the invention includes an antibody as described in PCT/US2006/043690 (Fischkoff et al., WO/2007/056539).

Specific examples of an anti-CTLA4 antibody useful in the methods of the invention are Ipilimumab, a human anti-CTLA4 antibody, administered at a dose of, for example, about 10 mg/kg, and Tremelimumab a human anti-CTLA4 antibody, administered at a dose of, for example, about 15 mg/kg. See also Sammartino, et al., Clinical Kidney Journal, 3(2):135-137 (2010), published online December 2009.

In other embodiments, the antagonist is a small molecule. A series of small organic compounds have been shown to bind to the B7-1 ligand to prevent binding to CTLA4 (see Erbe et al., J. Biol. Chem., 277:7363-7368 (2002). Such small organics could be administered alone or together with an anti-CTLA4 antibody to reduce inhibitory signal transduction of T cells.

### c. Other Immune Checkpoint Inhibitors

In another embodiment, the activatable drug delivery agent can contain an immune checkpoint inhibitor that inhibits the activity of other immune checkpoint molecules such as but not limited to B7-H3, B7-H4, BTLA, IDO, KIR, LAG3, NOX2, TIM3, VISTA, SIGLEC7, and SIGLEC9.

B7-H3, also known as CD276, is an immune checkpoint molecule from the B7 family. B7-H3 participates in the regulation of T-cell-mediated immune response. It also plays a protective role in tumor cells by inhibiting natural-killer mediated cell lysis as well as a role of marker for detection of neuroblastoma cells. It is also involved in the development of acute and chronic transplant rejection and in the regulation of lymphocytic activity at mucosal surfaces. B7-H3 immunotherapeutic agents are known in the art. Exemplary anti-B7-H4 agents include, but are not limited to, those described in the following U.S. Pat. Nos. 7,847,081, 8,802,091, and 9,371,395, all of which are specifically incorporated by reference herein in their entirety.

Indoleamine 2,3-dioxygenase(IDO), is a tryptophan catabolic enzyme with immune-inhibitory properties. IDO is known to suppress T and NK cells, generate and activate Tregs and myeloid-derived suppressor cells, and promote tumor angiogenesis. IDO immunotherapeutic agents are known in the art. Exemplary anti-IDO agents include, but are not limited to, those described in the following U.S. Pat. Nos. 7,598,287, 9,598,422, and 10,323,004, all of which are specifically incorporated by reference herein in their entirety.

Lymphocyte Activation Gene-3 (LAG3) is an inhibitory receptor on antigen activated T-cells. LAG3 delivers inhibitory signals upon binding to ligands, such as FGL1. Following TCR engagement, LAG3 associates with CD3-TCR in the immunological synapse and directly inhibits T-cell activation. LAG3 suppresses immune responses by action on Tregs as well as direct effects on CD8+ T cells. LAG3 immunotherapeutic agents are known in the art. Exemplary anti-LAG3 agents include, but are not limited to, those described in the following U.S. Pat. Nos. 10, 188,730 and 10,358,495, both of which are specifically incorporated by reference herein in their entirety.

V-type immunoglobulin domain-containing suppressor of T-cell activation (VISTA) is an immunoregulatory receptor which inhibits the T-cell response. VISTA is expressed on hematopoietic cells. VISTA immunotherapeutic agents are known in the art. Exemplary anti-VISTA agents include, but are not limited to, those described in the following U.S. Pat. Nos. 9,381,244 and 10,273,301, both of which are specifically incorporated by reference herein in their entirety.

### d. Antimicrobials

In other embodiments, activatable drug delivery agent include an antimicrobial agent within their core. For example, the antimicrobial can be an antibiotic, an antifungal, an antiviral, an antiparasitics, or essential oil.

### B. Logic Gates

In contrast to classical binary bits such as those in the biological ADC described above, the use of protease activity as probabilistic bits is also disclosed herein. There is abundant evidence that many biological phenomena are stochastic events, including chemical reactions, gene expression, and enzymatic activity. By leveraging both multi-target and common-target protease promiscuity, logic gates as disclosed here to operate on the probability states of two-state proteases to provide the ability to solve inference-based oracle problems, such as LPN, by deducing the correct value of hidden strings with the highest probability.

In the noise-free parity learning problem, an oracle is hiding a string, a, of bits (e.g., a=[01], a=[11], etc.), and the goal is to infer the value of the hidden string. The user can make “oracle queries”, which cause the oracle to generate random strings, x, and calculate the dot product (i.e., the scalar product of two vectors) between the hidden string and the random string (i.e., a·x) to produce an answer bit. By repeating this process, the user creates a log of calculations where the randomly generated string and the answer bit are known, and this information is used to infer the identity of the hidden string. By contrast, in the Learning Parity with Noise problem, either environmental or systemic (i.e., a faulty oracle) sources of noise cause the parity between the bits in the hidden string and the answer bit to be <100%. The LPN problem is more relevant to biological systems, which exhibit many different types of noise, including enzyme promiscuity.

One embodiment provides a set of probabilistic gates to perform operations on the state probabilities of two-state (i.e., state 0 or state 1) proteases. In one embodiment, there are two probabilistic gates, the Uniform Gate (U-gate) and the Linker Gate (L-gate). The Uniform Gate takes an input protease, b₀, with a state 0 or 1 probability of 100%, and creates an equal superposition of states by outputting b₀ in state 0 and 1 with 50% probability each. The Linker Gate is analogous to a classical XOR gate, more specifically the L-Gate links, or matches, the state 1 probabilities of two proteases to the same value. The L-gate takes a control and target protease (b₀ and b₁, respectively) and operates on the state 1 output probability of target b₁ to match with the state 1 probability of control b₀ at a user-defined value that can take a probability between 0.5 and 1.0 to control the strength of the match between the proteases (i.e., tune the likelihood that both proteases would be found in state 1.0 simultaneously). This operation is applied if and only if the state 1 input probability of control b₀ is nonzero, otherwise, the state 1 output probability of b₁ remains unchanged.

The following are rules that can be applied to the disclosed logic gates. To use a notation that can account for a gate, GATE, that processes multiple bits (i.e., n total bits), included is a subscript that identifies the protease (bit), bₙ, a superscript that identifies whether the value is an input (i.e., “IN”) or an output (i.e., “OUT”), and a number within parentheses that denotes the state (i.e., state-0 or state-1).

GATE(p_{b}_{ₙ}^{IN}(0), p_{b}_{ₙ}^{IN}(1))=(p_{b}_{ₙ}^{OUT}(0), p_{b}_{ₙ}^{OUT}(1))

Using this notation, the first possible case of operation for the U-gate (i.e., uniform gate) is to create a uniform superposition of the 0-state and the 1-state, if the input occupies only one state. To initially pass through a U-gate, the input protease (bit) must be initialized to be occupying only the 0 state or the 1 state: U(1.0, 0.0)=(0.5, 0.5). Or alternatively, U(0.0, 1.0)=(0.5, 0.5).

By contrast, the second case, which embodies the reversibility of this gate, applies when two U-gates are applied sequentially (with no gates in between). In this case, the effect of the U-gate is reversed, and the original input is returned. For one example, if the input into the first U-gate was (1.0, 0.0), then the output of the second U-gate would be (1.0, 0.0):

if: U₁(0.0, 1.0)=(0.5, 0.5)

then: U₂(0.5, 0.5)=(1.0, 0.0)

Alternatively, if the input into the first U-gate was (0.0, 1.0), then the output of the second U-gate would be (0.0, 1.0):

if: U₁(0.0, 1.0)=(0.5, 0.5)

then: U₂(0.5, 0.5)=(0.0, 1.0)

For all other cases of input probabilities where the probability of occupying the 0 state, x, does not equal 0.5, then the output probability is the same as the input:

if: x≠0.5

then: U(x, 1−x)=(x, 1−x)

In one embodiment, this occurs when a different gate (i.e., L-gate) is applied before the U-gate.

By contrast, the L-gate operates on two input proteases (bits), b₀, the control protease (bits), and b₁, the target bit, each with an associated probability distribution, and operates in two possible cases. In the first case, when the probability, y, of the control protease (bit) occupying the 0-state does not equal 1.0, then the output 0-state probability, z, of the target protease (bit) will be altered to match that of the control protease (bit):

if: y≠1.0

then (b₀): L(y, 1−y)=(y′, 1−y′)

then (b₁): L(z, 1−z)=(y′, 1−y′)

Otherwise, in all cases where y=1.0, then the target protease (bit), b₁, is unchanged:

if: y=1.0

then (b₀): L(1.0, 0.0)=(1.0, 0.0)

then (b₁): L(z, 1−z)=(z, 1−z)

To solve the oracle scores, each U- or L-gate operation is applied in succession to generate final state 0 and 1 output probabilities for all three protease bits. By multiplying all permutations of the output state 0 and 1 probabilities of proteases b₀-b₂ to estimate the joint probabilities the disclosed logic gates can correctly deduced the value of the hidden string among all other possibilities by assigning it the highest probability in all four oracle configurations.

### C. Computer-Implementation

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, and the like.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

Thus, another aspect of the disclosure is a system that is capable of carrying out a part or all of a method of the disclosure, or carrying out a variation of a method of the disclosure as described herein in greater detail. Exemplary systems include, as one or more components, computing systems, environments, and/or configurations that may be suitable for use with the methods and include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like. In some variations, a system of the disclosure includes one or more machines used for analysis of biological material (e.g., biological samples or specimen), as described herein. In some variations, the analysis of the biological material involves a machine capable of detecting and quantifying fluorescent signals.

The computer may operate in a networked environment using logical connections to one or more remote computers, such as a remote computer via a network interface controller (NIC). The remote computer may be a personal computer, a server, a router, a network PC, a peer device or other common network node, and typically includes many or all of the elements described above relative to the computer. The logical connection between the NIC and the remote computer may include a local area network (LAN), a wide area network (WAN), or both, but may also include other networks. Such networking environments are commonplace in offices, enterprise-wide computer networks, intranets, and the Internet. The remote computer may also represent a web server supporting interactive sessions with the computer; or in the specific case of location-based applications may be a location server or an application server.

### III. Methods of Use

The disclosed systems and methods can be used for the detection and quantification of protease activity in a biological sample from a subject suspected of having a disease characterized with aberrant protease signaling. The systems and methods can additionally be used to deliver regulated amounts of therapeutics to the subject to treat disease.

The dysregulated activity of protease networks is a hallmark of complex diseases which has provided the impetus to use measurements of protease activity as biomarkers of disease and disease severity. Because of their ability to alter proteins, proteases are involved in regulating the fate, localization, and activity of proteins, modulating protein-protein interactions, creating new bioactive molecules, contributing to the processing of cellular information, and generating, transducing, and amplifying molecular signals. Proteases can influence DNA replication and transcription, cell proliferation and differentiation, tissue morphogenesis and remodeling, heat shock and unfolded protein responses, angiogenesis, neurogenesis, ovulation, fertilization, wound repair, stem cell mobilization, hemostasis, blood coagulation, inflammation, immunity, autophagy, senescence, necrosis, and apoptosis. Alterations in proteolytic systems underlie the pathogenesis of numerous diseases such as but not limited to cancer, neurodegenerative diseases, inflammatory diseases, and cardiovascular diseases. The systems and methods described herein leverage the activity of proteases to simultaneously detect proteases and deliver drugs to the subject in need thereof.

### A. Diagnosis

The disclosed biological circuits can be used to diagnose disease in a subject in need thereof. In one embodiment, the subject is suspected of having a disease or infection linked to aberrant protease signaling. In another embodiment, the subject is suspected of having a bacterial or viral infection with a bacteria or virus having membrane-embedded proteases. The analysis and detection of proteases in a biological sample can help with the diagnosis and subsequent treatment strategy of diseases having aberrant protease signaling.

In one embodiment, a sample is collected from a subject for analysis. The sample can be a biological sample such as but not limited to urine, blood, lymphatic fluid, plasma, saliva, or stool. In one embodiment, the selection of a sample type is determined by the disease or infection that the subject is suspected to have or has been diagnosed with previously. For example, a subject suspected of having a bacterial or viral infection could supply a blood sample.

Once the sample has been procured, it is contacted with a plurality of peptide caged biocomparators specific to the protease or proteases of interest. In some embodiments, the plurality of peptide caged biocomparators contains biocomparators having different densities of peptides surrounding the core, such that the concentration of protease present in the biological sample will degrade and release the contents of biocomparators in order of magnitude. For example, a sample with a low concentration of the protease of interest will only be capable of opening biocomparators with a low density of peptide surrounding it. In this embodiment, each different biocomparator class (i.e. different peptide density) contains a unique signal molecule within its core so that the opened biocomparators can be detected within the sample. For example, a biocomparator having density A contains signal molecule 1, a biocomparator having density B contains signal molecule 2, etc. The detection of signal molecule 1 indicates that biocomparator A has been opened.

Each class of biocomparator has a threshold amount of protease activity necessary to cleave the peptide cage and open the biocomparator, releasing the signal cargo. The protease activity necessary for cleaving the peptide cages of the biocomparators is experimentally validated before exposing the biocomparators to the sample of interest or introducing the biocomparators into a subject. In such an embodiment, each biocomparator having a different density of peptide surrounding it, is exposed to increasing concentrations of the protease of interest. The concentration at which each biocomparator is opened is recorded as the threshold amount of protease required to open that specific biocomparator. Therefore, if a sample is contacted with a plurality of different biocomparators (surrounded by different densities of peptide and containing different cargo) the signal molecule detected in the sample will indicate the amount of protease present in the sample.

In another embodiment, after detection of the signal molecules, a binary digit is assigned to the sample, indicating the presence and/or relative amount of protease present in the sample. For example, an output of 00 would indicate the presence of little to no protease, 0, 1 and 1,0 would indicate the presence of an intermediate level of protease, and 1,1 would indicate the presence of protease over a threshold limit determined for the disease state. In such an embodiment, a sample having an output of 1,1 would indicate the subject likely has aberrant protease signaling and is diagnosed with disease. Diseases linked to aberrant protease signaling are detailed in a subsequent section below.

### B. Treatment

The disclosed drug delivery circuits can also be used for the treatment of diseases and disorders in subjects in need thereof. In one embodiment, the disease or disorders is characterized by aberrant protease signaling. In another embodiment, the disease is a bacterial or viral infection with a bacteria or virus having membrane-embedded proteases.

In one embodiment, the subject is first administered a plurality of peptide caged biocomparators specific to the protease or proteases of interest and a plurality of activatable drug delivery agents containing a therapeutic agent to treat the disease. The plurality of peptide caged biocomparators contain biocomparators having different densities of peptides surrounding the core, such that the concentration of protease present in the subject will degrade and release the contents of biocomparators in order of magnitude. For example, a sample with a low concentration of the protease of interest will only be capable of opening biocomparators with a low density of peptide surrounding it. In this embodiment, each different biocomparator class (i.e. different peptide density) contains unique cargo within its core such that the amount of protease present in the subject will open biocomparators containing cargo to activate the drug delivery agent containing the amount drug necessary to treat the disease or kill the bacteria. In some embodiments, the drug delivery agents are activatable by cleavage of a cleaved peptide or polymer surrounding the therapeutic agent. The peptide can be cleaved by proteases released by the biocomparator. In some embodiments, the biocomparators also contain protease inhibitors such that proteases are inhibited and the biocomparator can control the drug delivery agent that is activated.

Each class of biocomparator has a threshold amount of protease activity necessary to cleave the peptide cage and open the biocomparator, releasing the cargo. The protease activity necessary for cleaving the peptide cages of the biocomparators is experimentally validated before exposing the biocomparators to the sample of interest or introducing the biocomparators into a subject. In such an embodiment, each biocomparator having a different density of peptide surrounding it, is exposed to increasing concentrations of the protease of interest. The concentration at which each biocomparator is opened is recorded as the threshold amount of protease required to open that specific biocomparator. In one embodiment, biocomparators are loaded with cargo specific to the protease concentration necessary to open the appropriate activatable drug delivery agent. In such embodiments, biocomparators requiring a lower concentration of protease to cleave the peptide cage activate a drug delivery agent having a lower drug dose, whereas biocomparators requiring a higher concentration of protease to cleave the peptide cage activate a drug delivery agent having a higher drug dose. Therefore, if a subject is administered a plurality of different biocomparators (surrounded by different densities of peptide and containing different cargo) the peptides within the subject will only open biocomparators, if the proteases exceed the threshold activity necessary to open them, and those biocomparators will activate only the drug delivery agent having the minimum amount of therapeutic agent necessary to treat the disease or disorder. Therefore, the proteases within the subject serve to both release and titrate drug delivery within the subject. The biocomparators and drug delivery agents that are not opened will be filtered and excreted.

### 1. Subjects to be Treated

### a. Bacterial or Viral Infection

The disclosed systems and methods can be used to treat infections and infectious diseases. Bacterial and viral cells commonly contain membrane-embedded proteases that can be targeted using the disclosed systems and methods.

The infection or disease can be caused by a bacterium, virus, protozoan, helminth, or other microbial pathogen that enters intracellularly and is attacked, i.e., by cytotoxic T lymphocytes.

The infection or disease can be acute or chronic. An acute infection is typically an infection of short duration. During an acute microbial infection, immune cells begin expressing immunomodulatory receptors. Accordingly, in some embodiments, the method includes increasing an immune stimulatory response against an acute infection.

The infection can be caused by, for example, but not limited to Candida albicans, Listeria monocytogenes, Streptococcus pyogenes, Streptococcus pneumoniae, Neisseria meningitidis, Staphylococcus aureus, Escherichia coli, Acinetobacter baumannii, Pseudomonas aeruginosa or Mycobacterium.

In some embodiments, the disclosed systems and methods are used to treat chronic infections, for example infections in which T cell exhaustion or T cell anergy has occurred causing the infection to remain with the host over a prolonged period of time.

Exemplary infections to be treated are chronic infections cause by a hepatitis virus, a human immunodeficiency virus (HIV), a human T-lymphotrophic virus (HTLV), a herpes virus, an Epstein-Barr virus, or a human papilloma virus.

Representative infections that can be treated, include but are not limited to infections cause by microorganisms including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, Yersinia, Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni.

Other microorganisms that can be treated using the disclosed compositions and methods include, bacteria, such as those of Klebsiella, Serratia, Pasteurella; pathogens associated with cholera, tetanus, botulism, anthrax, plague, and Lyme disease; or fungal or parasitic pathogens, such as Candida (albicans, krusei, glabrata, tropicalis, etc.), Cryptococcus, Aspergillus (fumigatus, niger, etc.), Genus Mucorales (mucor, absidia, rhizophus), Sporothrix (schenkii), Blastomyces (dermatitidis), Paracoccidioides (brasiliensis), Coccidioides (immitis) and Histoplasma (capsulatuma), Entamoeba, histolytica, Balantidium coli, Naegleria fowleri, Acanthamoeba sp., Giardia Zambia, Cryptosporidium sp., Pneumocystis carinii, Plasmodium vivax, Babesia microti, Trypanosoma brucei, Trypanosoma cruzi, Toxoplasma gondi, etc.), Sporothrix, Blastomyces, Paracoccidioides, Coccidioides, Histoplasma, Entamoeba, Histolytica, Balantidium, Naegleria, Acanthamoeba, Giardia, Cryptosporidium, Pneumocystis, Plasmodium, Babesia, or Trypanosoma, etc.

### b. Cancer

The disclosed systems and methods can be used to treat cancer. Protease signaling is dysregulated in some cancers. Cancer cells acquire a characteristic set of functional capabilities during their development through various mechanisms. Aberrant protease signaling (amplified, diminished activity, or altered localization) can contribute to many of these so called hallmark capabilities of cancer. Such capabilities include evading apoptosis, self-sufficiency in growth signals, insensitivity to anti-growth signals, tissue invasion/metastasis, limitless replicative potential, inflammation, immune evasion, and sustained angiogenesis.

The term “cancer cell” is meant to encompass both pre-malignant and malignant cancer cells. In some embodiments, cancer refers to a benign tumor, which has remained localized. In other embodiments, cancer refers to a malignant tumor, which has invaded and destroyed neighboring body structures and spread to distant sites. In yet other embodiments, the cancer is associated with a specific cancer antigen (e.g., pan-carcinoma antigen (KS ¼), ovarian carcinoma antigen (CA125), prostate specific antigen (PSA), carcinoembryonic antigen (CEA), CD19, CD20, HER2/neu, etc.).

The systems and methods disclosed herein are useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xenoderma pegmentosum, keratoactanthoma, seminoma, thyroid follicular cancer and teratocarcinoma.

Cancers caused by aberrations in apoptosis can also be treated by the disclosed systems and methods. Such cancers may include, but are not be limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions in the ovary, bladder, breast, colon, lung, skin, pancreas, or uterus. In other specific embodiments, sarcoma, melanoma, or leukemia is treated or prevented by the methods and compositions.

Specific cancers and related disorders that can be treated or prevented by systems and methods disclosed herein include, but are not limited to, leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as, but not limited to, Hodgkin's disease or non-Hodgkin's disease lymphomas (e.g., diffuse anaplastic lymphoma kinase (ALK) negative, large B-cell lymphoma (DLBCL); diffuse anaplastic lymphoma kinase (ALK) positive, large B-cell lymphoma (DLBCL); anaplastic lymphoma kinase (ALK) positive, ALK+anaplastic large-cell lymphoma (ALCL), acute myeloid lymphoma (AML)); multiple myelomas such as, but not limited to, smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including but not limited to, pheochromocytom and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, acromegaly, and diabetes insipius; eye cancers including, but not limited to, ocular melanoma such as iris melanoma, choroidal melanoma, and cilliary body melanoma, and retinoblastoma; vaginal cancers, including, but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

### c. Inflammatory and Autoimmune Diseases

In one embodiment, the subject has an inflammatory or autoimmune disease. Representative inflammatory or autoimmune diseases/disorders include, but are not limited to, rheumatoid arthritis, systemic lupus erythematosus, alopecia areata, ankylosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis - juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

In some embodiments the inflammation or autoimmune disease is caused by a pathogen, or is the result of an infection.

### EXAMPLES

### Example 1. A Biological ADC Converts Protease Activity to Classical Bits

### Materials and Methods

Protease cleavage assays: All protease cleavage assays were performed with a BioTek Cytation 5 Imaging Plate Reader, taking fluorescent measurements at 485/528 nm and 540/575 nm (excitation/emission) for read-outs measuring peptide substrates terminated with FITC (Fluorescein isothiocyanate) and 5-TAMRA (5-Carboxytetramethylrhodamine), respectively. Kinetic measurements were taken every minute over the course of 60-120 minutes at 37° C. West Nile Virus NS3 protease (WNVp) and Tobacco Etch Virus protease (TEVp), along with their substrates, inhibitors and buffers were obtained from Anaspec, Inc. (Fremont, Calif.). Phospholipase C (PLC), Phosphatidylinositol-Specific (from Bacillus cereus) was purchased from Thermo Fisher Scientific (Waltham, Mass.). Activity RFU measurements were normalized to time 0 measurement, and as such represent fold change in signal. Granzyme B (GzmB) was purchased from PeproTech, Inc. (Rocky Hill, N.J.). Thrombin and Factor XIa were purchased from Haematologic Technologies (Essex, Vt.). Outer Membrane Protease T (OmpT, Protease 7) was purchased from Lifespan Biosciences (Seattle, Wash.). C1r was purchased from Millipore Sigma (Burlington, Mass.). GzmB, Thrombin, Factor XIa, and C1r fluorescent peptide substrates were custom ordered from CPC Scientific (Sunnyvale, Calif.). OmpT fluorescent peptide substrate was custom ordered from Genscript (Piscataway, N.J.). See Table 1 and Table 2 for more information regarding proteases, substrates, and inhibitors.

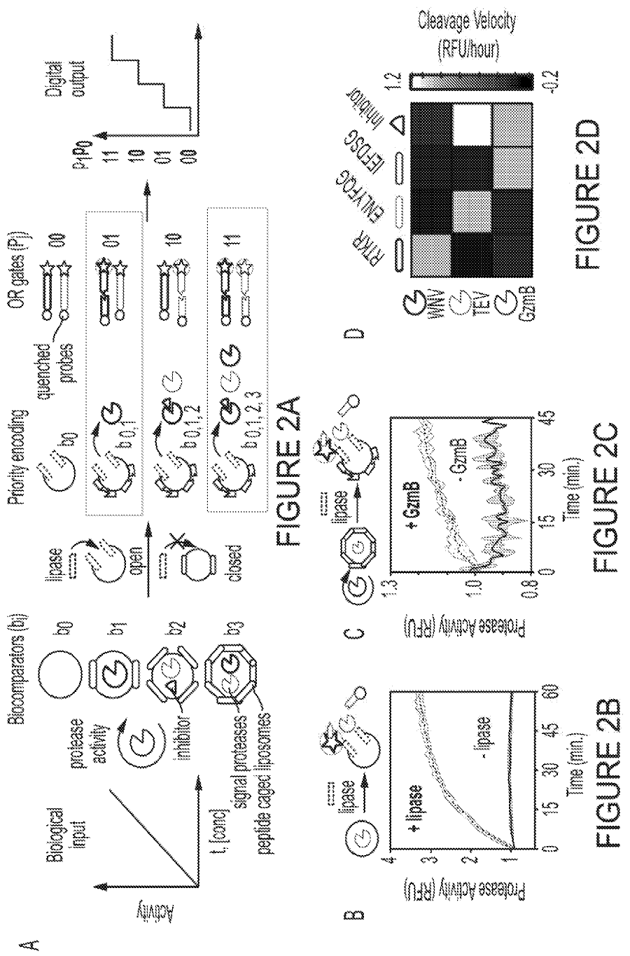

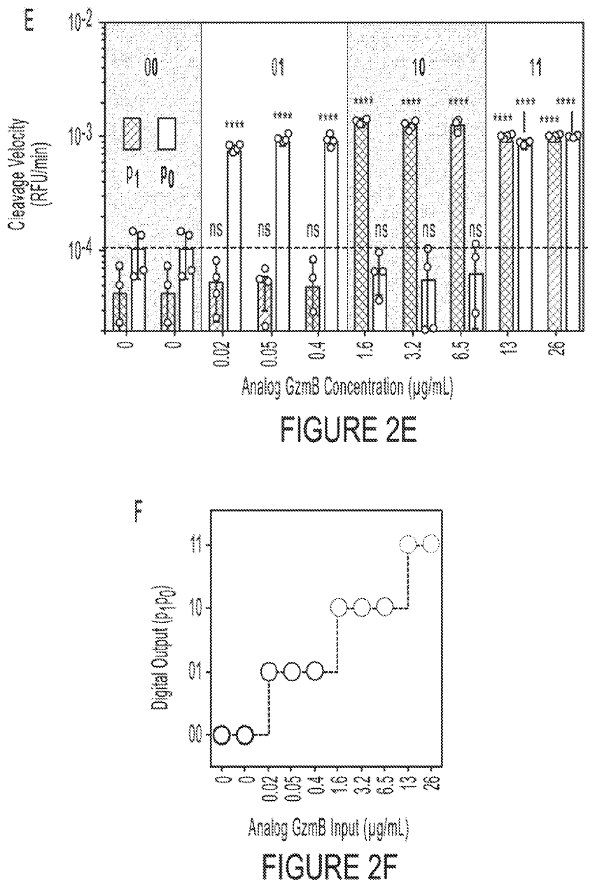

For FIG. 2B: 10 uL of liposomes (34 mM lipids) loaded with TEVp (1 ug protease/17 mmol liposome) were coincubated with 50 uL TEVp substrate in provided activity buffer (pH 7.5). 2 uL of PLC (100 units/mL) was added to the experimental group, and 2 uL of assay buffer was added to the control group.

For FIG. 2C: 10 uL of liposomes (34 mM lipids) loaded with TEVp (1 ug protease/17 mmol liposome), embedded with 10 mol % CPAA and crosslinked at 0.1% efficiency with GzmB substrate were coincubated with 50 uL TEVp substrate in provided activity buffer (pH 7.5). 2 uL PLC (100 U/mL) was added to both the control and experimental group. 2 uL GzmB (0.1 ug/uL) was added only to experimental group.

For FIG. 2D: All amounts of protease, substrate, and inhibitor for WNVp and TEVp were added according to instructions from Anaspec WNVp and TEVp activity kit. All conditions incubated with WNVp inhibitor include protease of interest incubated with its primary substrate. GzmB was added at a working concentration of (0.01 mg/mL) to 2 uM of its peptide substrate.

For FIG. 2E: All 4 biocomparator levels (b₀-b₃, 50 mM lipids each) were added together (10 uL each), and co-incubated with 13 uL of GzmB solution (concentration varies depending on condition), 2 uL of PLC (100 U/mL), 0.5 uL of WNVp substrate (after diluted 100× according to manufacturer's instructions), 0.5 uL of TEVp substrate (after diluted 100× according to manufacturer's instructions), and 4 uL of assay buffer. Biocomparator levels 0-3 are referenced by peptide cage crosslinking efficiencies of 0, 0.01, 1 and 100%, respectively. Plotted values are taken at minute 30 and normalized to starting values (time 0, or equivalently, the no protease control). Unpaired, one-way t-tests (n=4) were performed between the condition with GzmB and the negative control (no GzmB) for each respective output (i.e., p₀ or p₁).

TABLE 1
Listing of peptides.

| | | SEQ |
|---|---|---|
| | | ID |
| Name | Peptide Sequence | NO: |
| GzmB linker | IEFDSGK | 6 |
| GzmBs | 5FAM-aIEFDSGK(CPQ₂)kkc | 7 |
| WNVs | 5TAMRA-RTKR(QXL570) | 8 |
| TEVs | 5FAM-ENLYFQG(QXL520) | 9 |
| OmpT linker | RRSRRVK | 10 |
| OmpTs | DABCYL-RRSRRV-K(5-FAM) | 11 |
| Polyarginine AMP | RRRRRRRR | 12 |
| Locked AMP p₀ | EEEEEEEEEEERKTRRRRRRRRR | 13 |
| Locked AMP p₁ | EEEEEEEEENLYFQGRRRRRRRRR | 14 |
| CC1 | 5FAM-LQRIYK-K(CPQ2)-C | 15 |
| CC2 | 5FAM-KSVARTLLYK-K(CPQ2)-C | 16 |
| CC4 | 5FAM-QRQRIIGG-K(CPQ2)-C | 17 |
| CC6 | 5FAM-KYLGRSYKV-K(CPQ2)-C | 18 |
| CC9 | 5FAM-GLQRALEI-K(CPQ2)-k-C | 19 |

TABLE 2
Listing of proteases and substrates.

| | | | SEQ ID |
|---|---|---|---|
| Protease Name | Abbreviation | Substrate(s) | NO: |
| Granzyme B | GzmB | 5FAM-aIEFDSGK(CPQ2)kkc, | 7 |
| | | IEFDSGK | 6 |
| West Nile Virus | WNVp | 5TAMRA-RTKR(QXL570), | 8 |
| Protease | | inhibitor: undeca-D-ArgNH₂ | |
| Tobacco Etch Virus | TEVp | 5FAM-ENLYFQG(QXL520) | 9 |
| Protease | | | |
| Outer Membrane | OmpT | DABCYL-RRSRRV-K(5-FAM) | 10 |
| Protein T | | | |
| Complement Protease | C1r | 5FAM-QRQRIIGG-K(CPQ2)-C, | 17 |
| C1r | | 5FAM-LQRIYK-K(CPQ2)-C | 15 |
| Thrombin | Thromb. | 5FAM-KSVARTLLVK-K(CPQ2)-C, | 16 |
| | | 5FAM-KYLGRSYKV-K(CPQ2)-C | 18 |
| Plasmin | Plasm. | 5FAM-GLQRALEI-K(CPQ2)-k-C, | 19 |
| | | 5FAM-KYLGRSYKV-K(CPQ2)-C | 18 |
| Factor XIa | Fac. XIa | 5FAM-LQRIYK-K(CPQ2)-C, | 15 |
| | | 5FAM-KYLGRSYKV-K(CPQ2)-C | 18 |

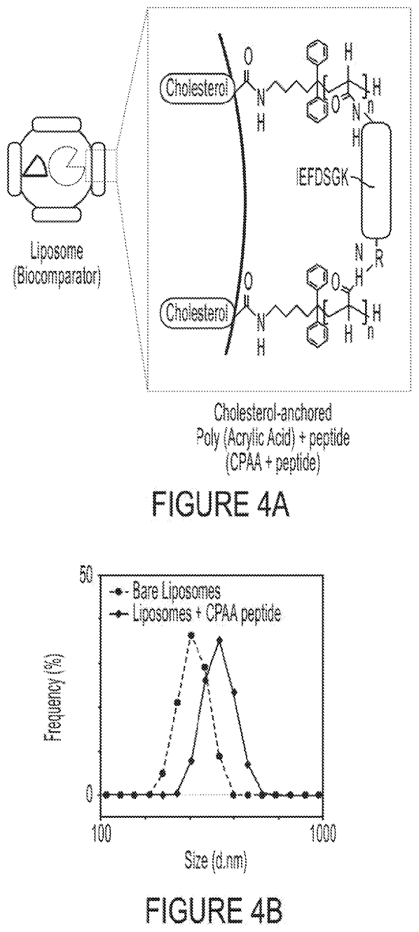

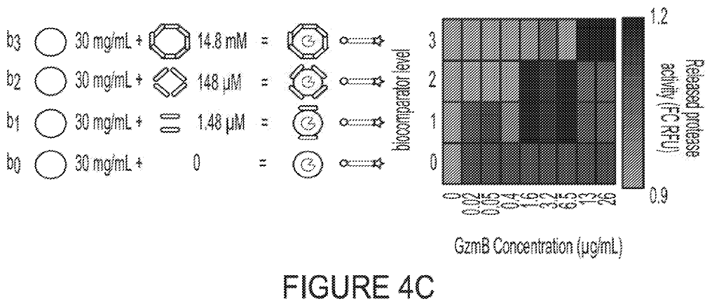

Liposome Synthesis and Characterization: Liposome synthesis kit, PIPES buffer, EDC*MeI, and spin filters (100 kDa m.w.c.o.) were purchased from Millipore Sigma (Burlington, Mass.). Cholesterol-anchored Polyacrylic Acid (4400 g/mol, 30-40 COOH groups/molecule, structure in Fig. S3A) was custom ordered from Nanocs (Boston, Mass.). Float-a-lyzer dialysis tubes (100 kDa m.w.c.o., 1 mL) were purchased from Spectrum Labs (Rancho Dominguez, Calif.). Synthesis protocol is adapted from the methods used by Basel et. al. Liposomes were loaded with respective protease inhibitor cocktail amounts, and concentration was estimated via absorbance. Standard curve for estimating concentration of liposomes was used by correlating absorbance of liposome solution at 230 nm with known standard concentrations (FIG. 4B). CPAA was vortexed in warm water (<10 mg/mL) and volume was added such that there was 10 mol % CPAA relative to the molarity of lipids in the liposome solution. Solution was incubated for 1 hour at room temperature, or overnight at 4 C. Excess polymer and materials were removed via centrifugation (spin filters, 3-5 times at 4700 XG for 10 mins) or float-a-lyzer membranes (4° C. in spinning water overnight). EDC*MeI was dissolved into 10 mM PIPES buffer and volume was added such that EDC*MeI:CPAA ratio was 4:1. Solution was incubated for 20 minutes at room temperature. Excess EDC was filtered out via centrifugation or dialysis tubes. Peptide crosslinker was added at desired molar ratio and incubated for 1 hour at room temperature or 4° C. Excess peptide was filtered via centrifugation or dialysis tubes. Change in liposome hydrodynamic diameter was measured via DLS on a Zetasizer Nano ZS, Malvern Panalytical (Netherlands). Volumes loaded into biocomparators include concentrations of proteases and inhibitors as follows: b₀=empty; b₁=20 uL WNVp (0.1 mg/mL)+80 uL DI H2O; b₂=50 uL WNV inhibitor (1 uM)+50 uL TEVp (0.04 mg/mL); b₃=50 uL WNVp (0.1 mg/mL)+50 uL TEVp (0.08 mg/mL).

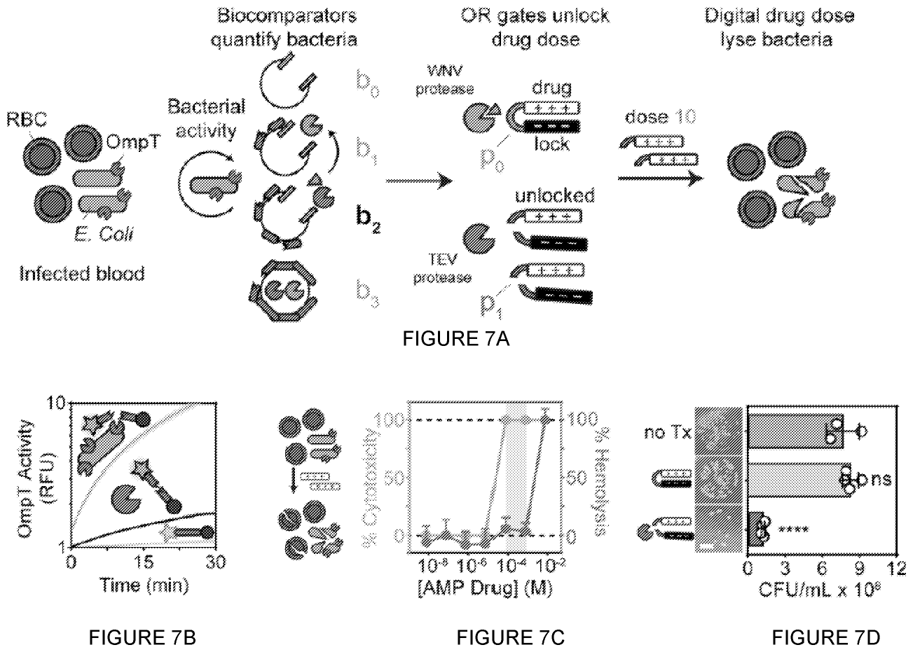

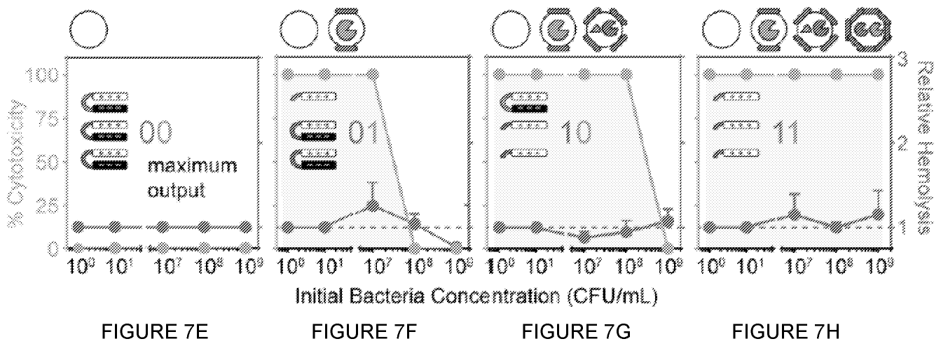

Statistical Analysis: Statistical analysis was performed using statistical packages included in GraphPad Prism 6. To assess the significance of increase in signal due to protease cleavage, a two-way ANOVA (without repeated measures) was used followed by Sidak's multiple comparisons test (FIG. 2B, 2C, and 7B). To assess the accuracy of assigning the binary value 0 or 1 to the digits p₀ and p₁ as seen in FIG. 2E, one-way unpaired t-tests were performed between the condition with GzmB and the negative control (no GzmB) for each respective output (i.e., p₀ or p₁). A one-way ANOVA followed by Dunnett's multiple comparisons test was used to compare experimental means to cells only control in FIG. 7D. Two-way ANOVA followed by Sidak's multiple comparisons test used to compare experimental means to control for bacterial cytotoxicity and RBC hemolysis (FIG. 7E-7H).

### Results

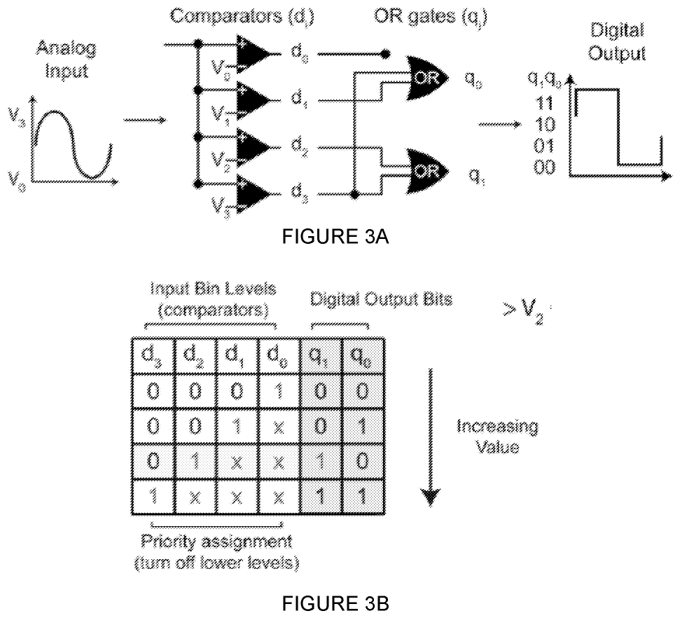

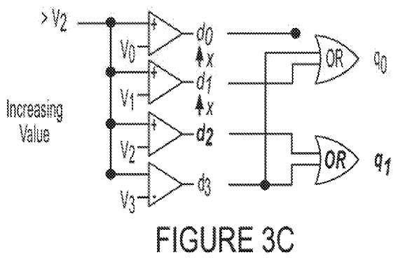

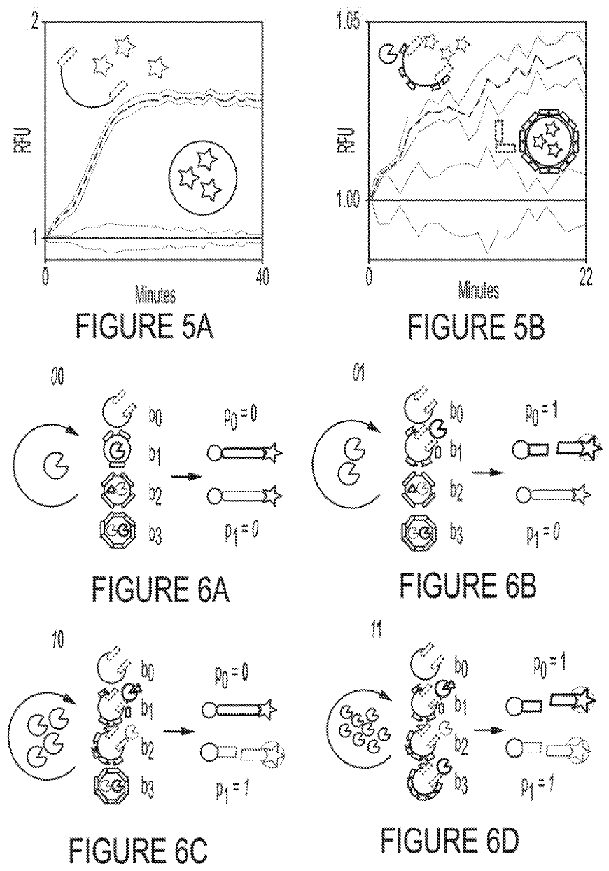

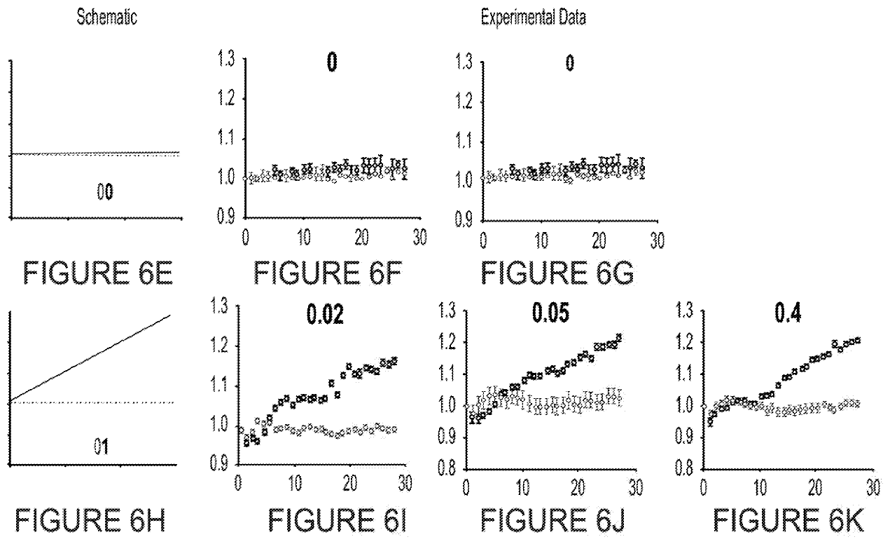

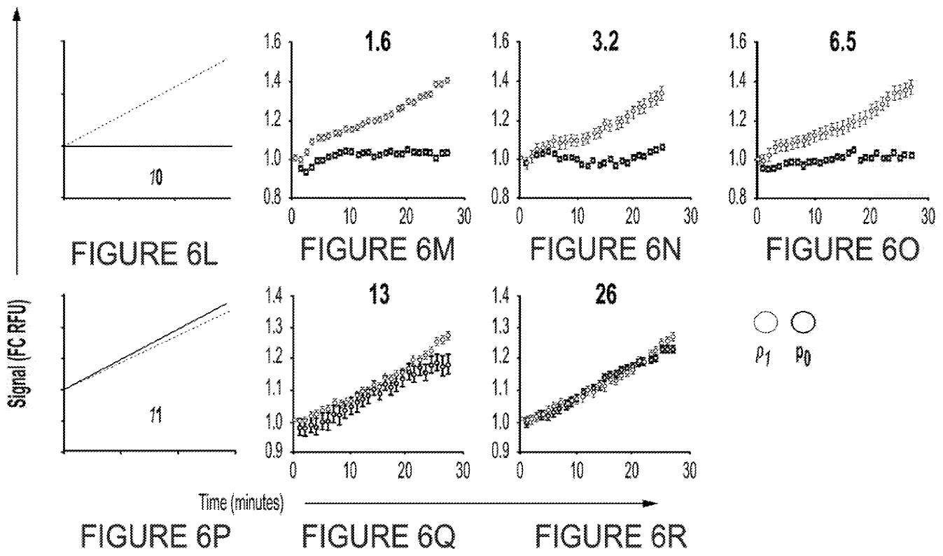

A central function of complex circuits is the ability to store and manipulate digitized information; therefore, a flash analog-to-digital converter (ADC) was constructed to convert continuous biological signals into binary digits. An electronic ADC performs three major operations during signal conversion: voltage comparison, priority assignment, and digital encoding. An analog input voltage is first compared against a set of increasing reference voltages (V₀-V) by individual comparators (d₀-dᵢ) that allow current to pass if the input signal is greater than or equal to its reference value (FIGS. 3A-3C). During priority assignment, only the activated comparator with the highest reference voltage, dₙ, remains on while all other activated comparators, dₙ₋₁-d₀ are turned off. The prioritized signal is then fed into a digital encoder comprising OR gates to produce binary values. To design an ADC biocircuit using protease activity as the core signal, biological analogs of comparators were constructed by using liposomes locked by an outer peptide cage (Basel, et al., ACS Nano, 5:2162-2175 (2011); Lee, et al., JACS, 129:15096-15097 (2007)) (FIGS. 2A, 4A, 4B). With increasing peptide crosslinking densities, these biocomparators (b₀-bᵢ) served to reference the level of input protease activity (GzmB) required to fully degrade the peptide cage (IEFDSGK, (SEQ ID NO:6), Table 1) and expose the lipid core (FIG. 4C), analogous to the reference voltages stored in electronic comparators. Lipase was used as a Buffer gate to open all biocomparators with fully degraded cages (FIGS. 2B, 2C; FIGS. 5A-5B) and release a unique combination of inhibitors and signal proteases (WNV, TEV, and WNV inhibitor) that collectively act to assign priority to the highest activated biocomparator (bₙ) by inhibiting all signal proteases released from other biocomparators (b₀-bₙ₋₁). To encode the prioritized signal into binary values, a set of OR gates were designed using orthogonal quenched substrates (RTKR (SEQ ID NO:8) and ENLYFQG (SEQ ID NO:9)) specific for the signal proteases (WNV and TEV respectively; FIG. 2D) to provide fluorescent 2-bit readouts (p₀-pᵢ; FIGS. 6A-6D). Fully integrated, our 4-2 bit biological ADC converted input protease levels (GzmB) across four orders of magnitude into binary digital outputs (FIGS. 2E-2F; FIGS. 6E-6R).

### Example 2. An Integrated BioADC to Execute an Antimicrobial Program

### Materials and Methods

Protease Cleavage Assays: See Example 1 for detailed overview of protease cleavage assay protocol. See below for specific details for each example.

For FIG. 3B: For recombinant OmpT condition, 2 μL of OmpT (0.5 mg/mL) was added to 18 μL of 2 μM OmpT substrate. For E. coli condition, 2 μL of E. coli (109 CFU/mL) was added to 18 μL of 2 μM OmpT substrate. 2 μL of DI H2o was added to negative control, along with 18 μL of OmpT substrate.

Bacterial Culture and Cytotoxicity Assay: DH5α Escherichia coli were cultured in LB broth (Lennox) at 37° C. and plated on LB agar (Lennox) plates. LB broth was purchased from Millipore Sigma (Burlington, Mass.) and LB agar was purchased from Invitrogen (Carlsbad, Calif.). AMP and locked AMP were custom ordered from Genscript (Piscataway, N.J.). See Table 1 for more information. Bacteria were grown to a concentration of 10⁹ CFU/mL before being used for experiments. Concentration was estimated by measuring the OD600 of the bacterial suspension, and assuming an OD600 of 1.000 corresponds to a concentration of 8×10⁸ CFU/mL. Bacterial cell viability was measured by making eight 10-fold serial dilutions, and plating three 10 μL spots on an LB agar plate. Plates were incubated overnight at 37° C., and CFUs were counted. Untreated bacteria CFU counts served as control for 0% cytotoxicity, and bacteria+IPA (or 0 countable CFUs) served as control for 100% cytotoxicity.

Red Blood Cell Hemolysis Assay: Healthy blood donors had abstained from aspirin in the last two weeks, and consent was obtained according to GT IRB H15258. Blood was drawn by median cubital venipuncture into sodium citrate (3.2%). The sample was subsequently centrifuged at 150 G for 15 min, and the resulting platelet rich plasma was discarded. Red blood cells were then washed three times with phosphate buffered saline (PBS). For each wash, 12 mL of PBS were added, the sample was centrifuged at 1000 RPM for 10 min, and the supernatant was discarded. Hemolysis was estimated by spinning down experimental RBC samples and measuring the absorbance of the supernatant at 450 nm. Absorbance values corresponding to 100% hemolysis came from incubating RBCs with 0.1% Tween-20. Absorbance corresponding to 0% hemolysis came from untreated RBCs.

For FIG. 7C: For bacterial cytotoxicity measurements, 25 μL of antimicrobial peptide (AMP) was added, pertaining to 7 concentrations ranging between 7.6 nM and 7.6 mM. 20 μL of bacteria (10⁷ CFU/mL) were added, and the sample was filled to 200 μL with LB broth in PCR tubes. Sample tubes were taped on a plate shaker (250 RPM) incubating at 37° C. for 8 hours. For RBC hemolysis measurements, the same assay was performed, but used 20 μL of donor RBCs instead of bacteria solution.

For FIG. 7D: For bacteria only condition, 5 μL of bacteria (10⁹ CFU/mL) were added to 95 μL LB broth. For bacteria+AMP p₁, 58 μL of AMP p₁ (1.7 mM) were added to 5 μL of bacteria (10⁹ CFU/mL), with the solution being filled to 100 μL with LB broth. For bacteria+protease+locked AMP p₁, 20 μL of TEVp (4 μg/mL) and 58 μL of AMP p₁ (1.7 mM) were added to 5 μL of bacteria (10⁹ CFU/mL), with the solution being filled to 100 μL with LB broth. Samples in PCR tubes were taped to a plate shaker (250 RPM) incubating at 37° C. for 1 hour. Serial dilutions and plating were then performed to measure viable bacteria concentrations.

For FIGS. 7E-7H: Each condition includes 20 μL of the bioprogram (2 μL of PLC, 6 μL D1, 6 μL D2, 6 μL D3), 20 μL of bacteria, 10 μL of RBCs, 24 μL of locked peptide drug (9 μL of 1.7 mM AMP p₁ and 15 μL of 0.53 mM AMP p₀), and 126 μL PBS. The concentration of bacteria, and the presence of each biocomparator, depends on the experimental condition. Samples in PCR tubes were taped to a plate shaker (250 RPM) incubating at 37° C. for 8 hours, followed by dilutions/plating to estimate bacterial cytotoxicity. The remainder of the sample was spun down by centrifugation and used to estimate hemolysis.

### Results

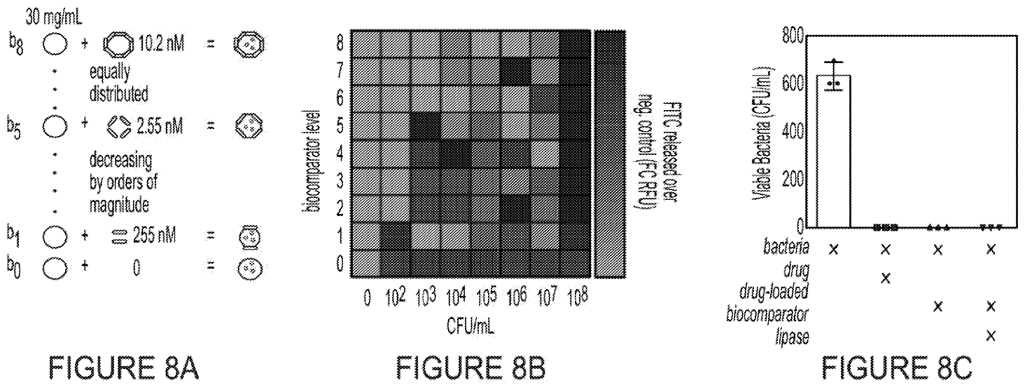

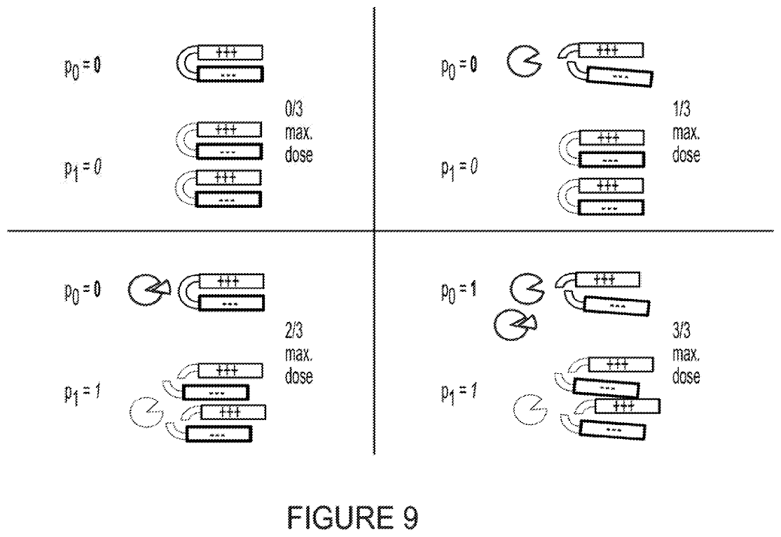

To demonstrate a practical biomedical application, the biological ADC was interfaced with a living system as a plug-and-play therapeutic biocircuit for digital drug delivery. The ADC was rewired to autonomously quantify input bacterial activity and then output an anti-microbial drug dose to selectively clear red blood cells (RBCs) of bacteria (DH5α Escherichia coli) (FIG. 7A). To construct biocomparators with the ability to prioritize input levels of bacterial activity, liposomes with peptide cages were synthesized using a substrate (RRSRRVK (SEQ ID NO:9)) specific for the E. coli surface protease OmpT (Grodberg, and Dunn, J Bacteriology, 170:1245-1253 (1988); McCarter, et al., J Bacteriology, 186:5919-5925 (2004)) (FIG. 7B). A series of 8 biocomparators with increasing peptide densities (i.e., peptide:liposome reaction ratios spanning 0, 8.5×10-3, 8.5×10⁻², 8.5×10⁻¹, 8.5, 85, 170, 255, 340 μmol/g) were synthesized and their ability to sense input bacterial concentrations was validated across 8 log units (0-10⁸ CFU/ml) using a fluorescent reporter (FIGS. 8A-8C). To convert the release of signal proteases to a drug output, protease-activatable prodrugs were designed using cationic (polyarginine) anti-microbial peptides (AMP) (FIG. 7C, Table 1) in charge complexation with anionic peptide locks (polyglutamic acid) to block the activity of AMP (Olson, et al., Integrative Biology, 1:382-393 (2009)). These drug-lock peptides were linked in tandem by OR gate peptides p₀ and p₁ (RTKR (SEQ ID NO:20) and ENLYFQG (SEQ ID NO:21) respectively) to allow signal proteases that directly cleave p₀ or p₁ to digitally control the output drug dose (FIG. 2A). One-third and two-thirds of the total drug dose was unlocked by cleavage of p₀ and p₁, respectively, such that binary values 00, 01, 10, and 11 corresponded to 0/3, ⅓, ⅔, and 3/3 of the total drug dose (FIG. 9).

To confirm the therapeutic efficacy of the prodrug design, treatment of bacteria with locked drug had no significant cytolytic activity compared to untreated controls, but by contrast, treatment with protease-cleaved drug-lock complexes resulted in a significant reduction in bacterial colonies (FIG. 7D). Similar levels of bacterial cytotoxicity were observed when AMP was directly loaded into liposomes, showing that charge complexation was required to fully block AMP activity (FIG. 8C). In human RBCs mixed with E. coli at concentrations ranging from 100-10⁹ CFU/mL, samples containing a single biocomparator (b₀) lacked the ability to eliminate bacteria as anticipated (output=00). By contrast, increasing the number of biocomparators in the samples (b₀-b₃) allowed our program to autonomously increase the drug dose (output 01, 10, and 11) in response to higher bacterial loads to completely eliminate infection burdens across 9 orders of magnitude up to 10⁹ CFU/mL without significantly increasing hemolysis (FIGS. 7E-7H). These data showed that cell-free biocircuits can be constructed using protease activity as a primary digital signal to execute autonomous drug delivery programs under a broad range of conditions.

### Example 3. Gillespie Model Validates Protease Cleavage Velocity as State Probabilities

### Results

In contrast to classical binary bits, the implications of quantifying protease activity as state probabilities were explored, because probabilistic circuits have been shown to efficiently solve inference-based problems (Kevin, M., Machine Learning: A Probabilistic Perspective, The MIT Press (2012)). At the single molecule level, individual enzymatic events have been shown to exhibit stochasticity that follow well-described statistical models (e.g., Poisson) (English, et al., Nature Chemical Biology, 2:168 (2006); Qian, H., Biophysical Journal, 95:10-17 (2008)). This has been exploited in past studies using restriction enzymes to perform stochastic computations where the probabilities for different outcomes were programmed by relative molar concentrations of different substrates (Adar, et al., PNAS, 101:9960-9965 (2004)). Therefore, it was reasoned that when a single promiscuous protease is exposed to two different substrates (e.g., substrate-0 or substrate-1), a single cleavage event of either substrate is stochastic, and with large number of proteases, the overall probabilities of the two possible states (i.e., a two-state protease bit cleaves substrate-0 or substrate-1) is directly correlated to the relative cleavage velocities measured in bulk for either substrate.

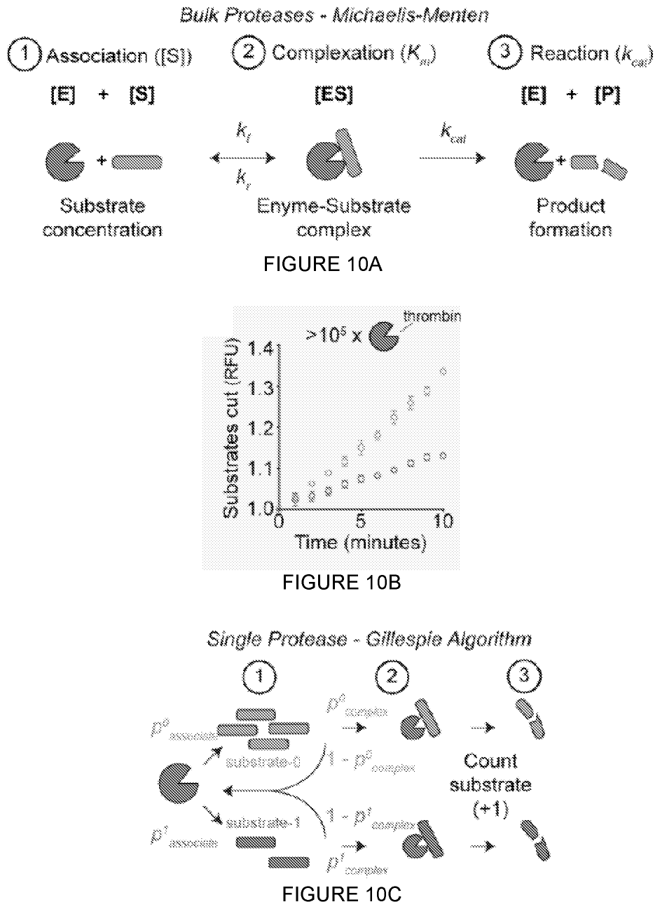

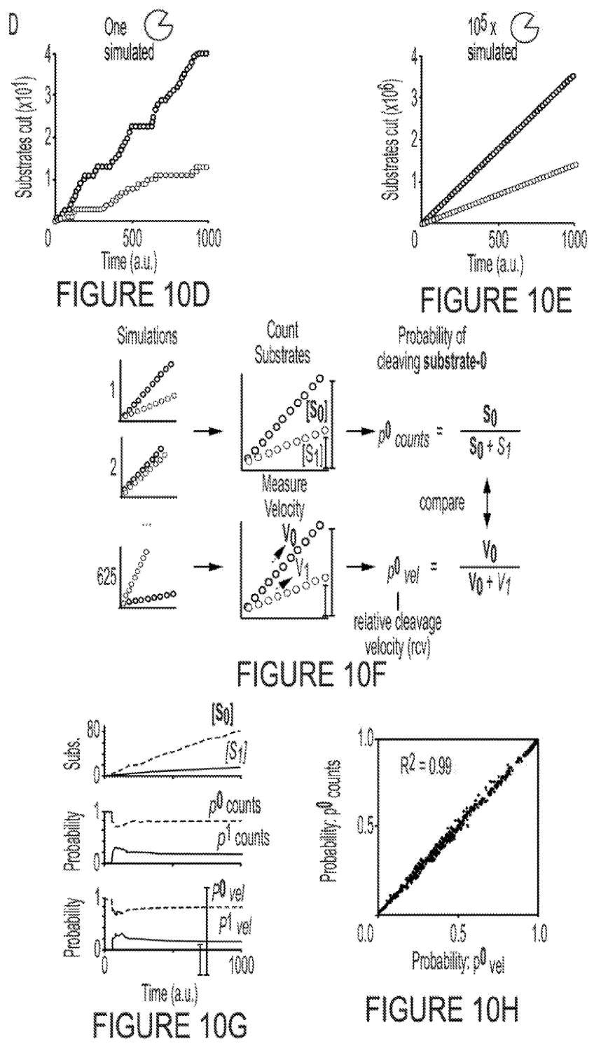

To validate this, the cleavage kinetics of a single protease were simulated by a Gillespie algorithm (Gillespie, D. T., J Physical Chemistry, 81:2340-2361 (1977); Gillespie, D. T., J Comp Phys, 22:403-434 (1976)), a well-established approach for modeling coupled chemical reactions and stochastic gene expression (Bratsun, et al., PNAS, 102:14593-14598 (2005)). This model consisted of three main phases that directly parallel Michaelis-Menten kinetics (FIGS. 10A-10B): (1) association between the protease and substrate-0 (blue) or substrate-1 (red) expressed as a probability based on relative substrate concentrations

$$( i . e . , {p}_{a s s o c i a t i o n}^{i} = \frac{[ {s}_{i} ]}{Σ [ {s}_{i} ]} , i = 0 , 1 ) , \qquad ( 2 )$$

successful complexation of the protease active site with the substrate (i.e., p_{complex}ⁱ) or failure (i.e., 1−p_{complex}ⁱ), and (3) catalytic cleavage of the substrate (i.e., k_{cat}) (FIG. 10C). In the single-protease simulation, stochastic, single-event increases in object counts (i.e., cleaved substrates) punctuated by periods of inactivity (FIG. 10D) were observed, which is consistent with single-enzyme studies that directly showed that periods between cleavage events (i.e., wait times) follow a probability distribution (English, et al., Nature Chemical Biology, 2:168 (2006); Qian, H., Biophysical Journal, 95:10-17 (2008)). By contrast, as the number of simulated proteases was increased to 10⁵ molecules, cumulative substrate cleavage events produced linear traces (FIG. 10E) that closely resembled experimental data of a prototypic protease (thrombin) cleaving two different substrates (i.e., a two-state bit) (FIG. 10B). To quantify the accuracy of using relative cleavage velocities to estimate the probability of protease cleavage by object counts, 625 distinct conditions were simulated using unique substrate concentrations (i.e., 10¹<[sᵢ]<10³) and complexation probabilities (i.e.0.1<p_{complex}ⁱ<1.0) (FIG. 10F). For each condition, the probability of the state 0 outcome (i.e., cleavage of substrate-0) was quantified by relative cleavage velocities (rcv)

$$( i . e . {p}_{velocity}^{0} = \frac{{v}_{o}}{Σ {v}_{i}} ) ,$$

and object counts

$$( ie . {p}_{c o u n t s}^{0} = \frac{[ {s}_{0} ]}{Σ [ {s}_{i} ]} ) ,$$

and found that after a period of time of initial fluctuation, both expressions converged to the same steady-state probability (FIG. 10G). Across all 625 unique conditions, a near-perfect correlation was found between pᵥₑₗ⁰ and p_{counts}⁰ therefore by the complement rule, pᵥₑₗ¹ and p_{counts}¹(R²=0.99, FIG. 10H). From these data, it was concluded that protease velocities are time-normalized equivalents to substrate counts (i.e., moles per unit time), and that relative cleavage velocities (rcv) can be used to estimate the state probability of a protease cutting a particular substrate.

### Example 4. Superposing Two-State bbits to Solve Oracle-Based Problems With Probabilistic Logic Gates

### Materials and Methods

Protease Cleavage Assays: See Example 1 for detailed overview of protease cleavage assay protocol. See below for specific details for each example.

For FIGS. 5G-5I and 5N-5Q: 2 uL of thrombin (10.1 mg/mL) or plasmin (6.9 mg/mL) were added to 18 uL of respective state 1 or state 0 substrate (2 uM). Velocities of Thrombin and Plasmin cutting their respective state 1 or state 0 substrates were calculated over the first 5 minutes of incubation. These velocities were normalized by the sum of the state 1 and state 0 velocity. Therefore, the values plotted as probability in FIG. 4 are relative velocities.

For FIG. 5S: Protease bbit configurations for each implementation of the Oracle are referenced in Table 1, and in each case 2 uL of protease was added to 2 uM of respective state substrate. U-gates are made reversible by adding in the original state substrate (state-0) at a concentration 10-fold the new state (state-1). Stock concentrations of the proteases involved were: factor XIa (6 mg/mL), plasmin (6.9 mg/mL), thrombin (10.1 mg/mL), and C1r (1 mg/mL).

State 0 and State 1 peptide substrates were CC1 and CC6 for FXIa, CC4 and CC1 for C1r, CC2 and CC6 for thrombin, and CC2 and CC9 for plasmin. Probability of two digit state is calculated by multiplying the probability (relative velocity) for each individual protease bbit. For example, if the first digit (bbit) is Protease A, with relative velocities Vₐ₁ and Vₐ₂, and the second digit is Protease B, with velocities V_{b1} and V_{b0}, then the probability of achieving the answer 01=V_{b0}*Vₐ₁.

Mathematical rules for probabilistic logic gates: To use a notation that can account for a gate, GATE, that processes multiple bits (i.e., n total bits), included is a subscript that identifies the protease (bit), bₙ, a superscript that identifies whether the value is an input (i.e., “IN”) or an output (i.e., “OUT”), and a number within parentheses that denotes the state (i.e., state-0 or state-1).

GATE(p_{b}_{ₙ}^{IN}(0), p_{b}_{ₙ}^{IN}(1))=(p_{b}_{ₙ}^{OUT}(0), p_{b}_{ₙ}^{OUT}(1))

Using this notation, the first possible case of operation for the U-gate (i.e., uniform gate) is to create a uniform superposition of the 0-state and the 1-state, if the input occupies only one state. To initially pass through a U-gate, the input bit must be initialized to occupying only the 0 state or the 1 state: U(1.0, 0.0)=(0.5, 0.5). Or alternatively, U(0.0, 1.0)=(0.5, 0.5).

By contrast, the second case, which embodies the reversibility of this gate, applies when two U-gates are applied sequentially (with no gates in between). In this case, the effect of the U-gate is reversed, and the original input is returned. For one example, if the input into the first U-gate was (1.0, 0.0), then the output of the second U-gate would be (1.0, 0.0):

if: U₁(0.0, 1.0)=(0.5, 0.5)

then: U₂(0.5, 0.5)=(1.0, 0.0)

Alternatively, if the input into the first U-gate was (0.0, 1.0), then the output of the second U-gate would be (0.0, 1.0):

if: U₁(0.0, 1.0)=(0.5, 0.5)

then: U₂(0.5, 0.5)=(0.0, 1.0)

For all other cases of input probabilities where the probability of occupying the 0 state, x, does not equal 0.5, then the output probability is the same as the input:

if: x≠0.5

then: U(x, 1−x)=(x, 1−x)

Experimentally, this occurs when a different gate (i.e., L-gate) was applied before the U-gate.

By contrast, the L-gate operates on two input bits, b₀, the control bit, and b₁, the target bit, each with an associated probability distribution, and operates in two possible cases. In the first case, when the probability, y, of the control bit occupying the 0-state does not equal 1.0, then the output 0-state probability, z, of the target bit will be altered to match that of the control bit:

if: y≠1.0

then (b₀): L(y, 1−y)=(y′, 1−y′)

then (b₁): L(z, 1−z)=(y′, 1−y′)

Otherwise, in all cases where y=1.0, then the target bit, b1, is unchanged:

if: y=1.0

then (b₀): L(1.0, 0.0)=(1.0, 0.0)

then (b₁): L(z, 1−z)=(z, 1−z)

### Results

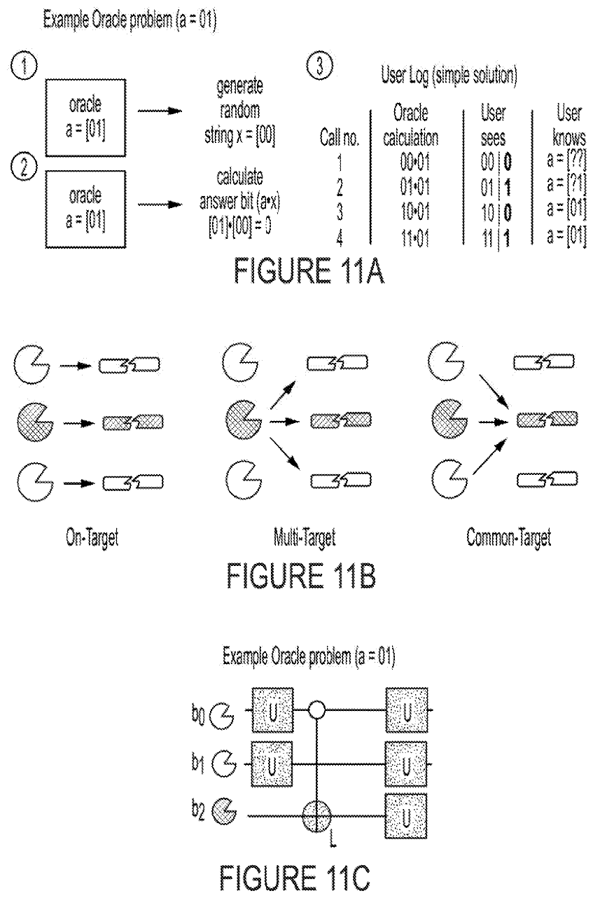

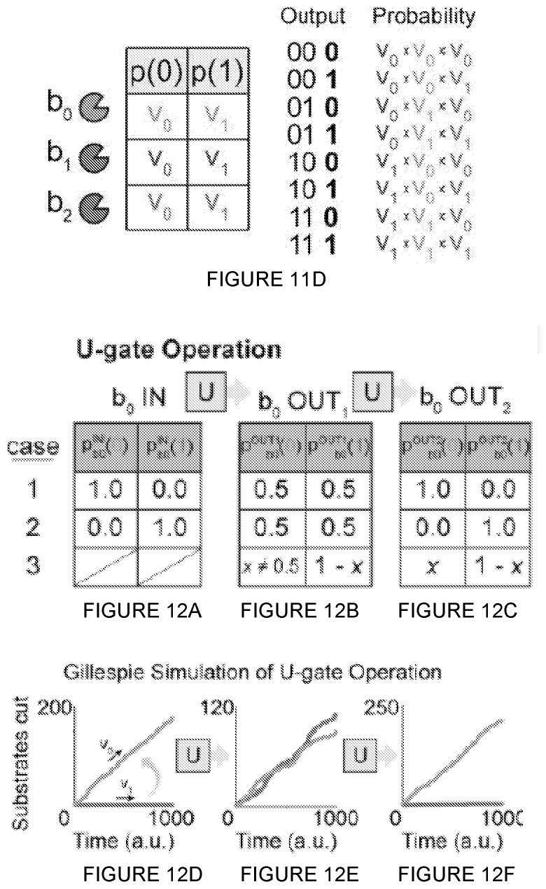

To demonstrate the use of protease activity as probabilistic bbits to solve inference problems, probabilistic circuits were designed to solve a classic oracle problem, Learning Parity with Noise (LPN) (Riste, et al., NP J Quantum Information, 3:1-5 (2017)). In the noise-free parity learning problem, an oracle is hiding a string, a, of bits (e.g., a=[01], a=[11], etc.), and the goal is to infer the value of the hidden string. The user can make “oracle queries”, which cause the oracle to generate random strings, x, and calculate the dot product (i.e., the scalar product of two vectors) between the hidden string and the random string (i.e., a·x) to produce an answer bit. By repeating this process, the user creates a log of calculations where the randomly generated string and the answer bit are known, and this information is used to infer the identity of the hidden string (FIG. 11A). By contrast, in the Learning Parity with Noise problem, either environmental or systemic (i.e., a faulty oracle) sources of noise cause the parity between the bits in the hidden string and the answer bit to be <100%. The LPN problem is more relevant to biological systems, which exhibit many different types of noise (Heams, et al., Mathematical Structures in Computer Science, 24:e240308 (2014)) including enzyme promiscuity (Lopez-Otin and Bond, J Biol Chem, 283:30433-30437 (2008)), so it was demonstrated that protease probabilities can be used to solve the LPN problem.

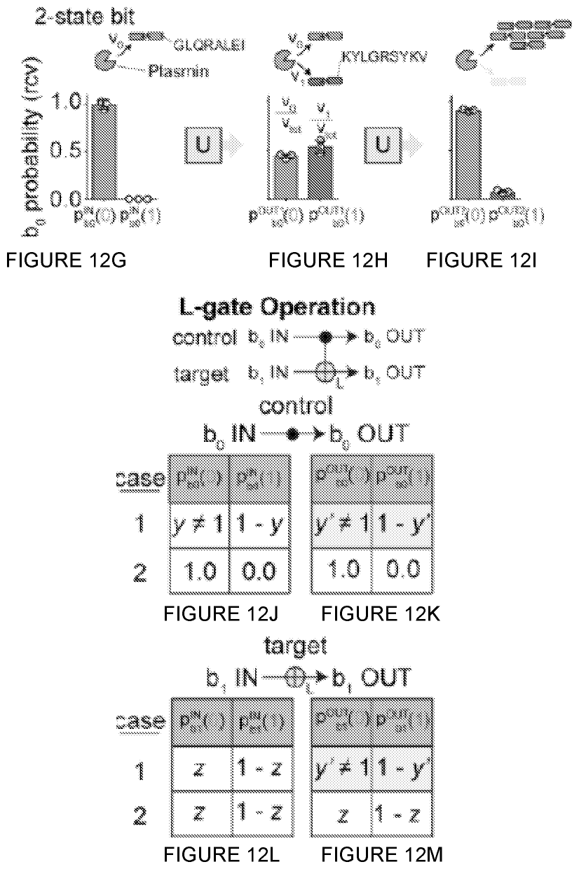

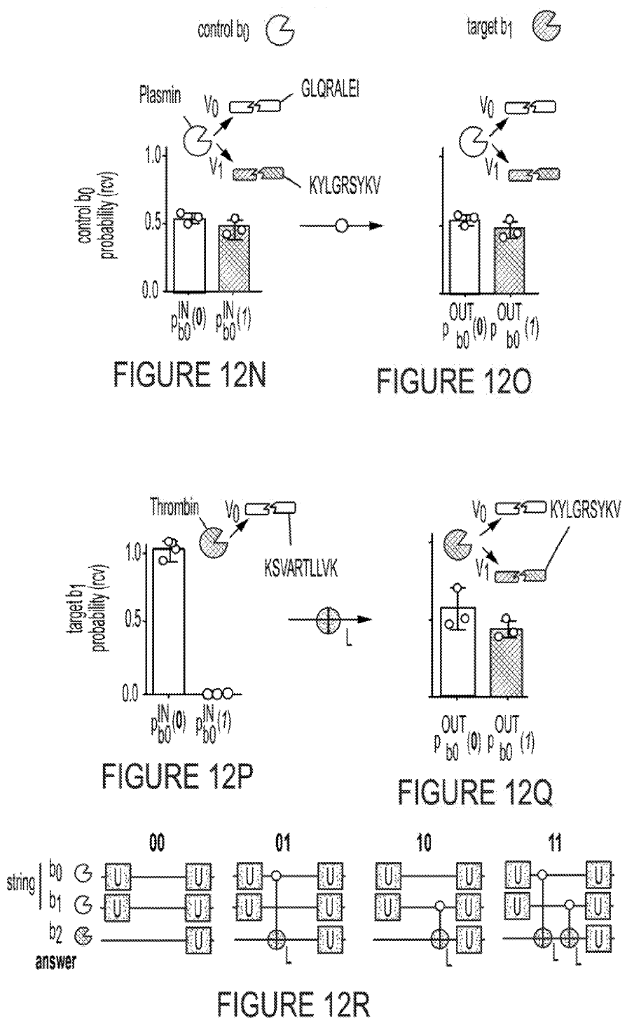

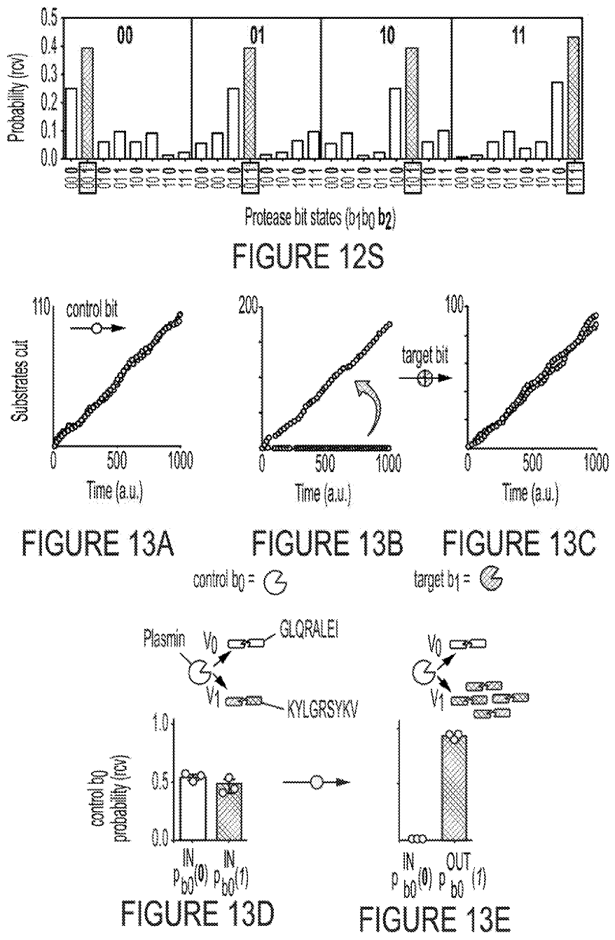

A set of probabilistic gates were built to perform operations on the state probabilities of two-state (i.e., state 0 or state 1) protease bits that were named the Uniform gate (U-gate) and Linker gate (L-gate). These gates make use of multi- and common-target promiscuity (FIG. 11B), and their operations were designed based on previous non-biological implementations of probabilistic gates that solved the LPN problem. Analogous to conducting a coin flip, the U-gate was designed to take an input protease bbit, b₀, with a state 0 or 1 probability of 100% (i.e., the only valid initial inputs are p_{b}_{₀}^{IN}(0)=1.0 or p_{b}_{₀}^{IN}(1)=1.0) and create an equal superposition of states by outputting b₀ in state 0 and 1 with 50% probability each (i.e. p_{b}_{₀}^{OUT}(0)=p_{b}_{₀}^{OUT}(1)=0.5) (FIGS. 12A-12C, case 1 & 2, OUT1). This was performed experimentally by taking a protease cleaving one substrate (e.g., substrate-0) and adding the complement substrate (e.g., substrate-1) at a molar concentration such that relative cleavage velocities (i.e., rcv) for both substrates were matched. The U-gate was further designed to be reversible such that two U-gates applied consecutively would revert b₀ back to its original state with 100% probability (FIGS. 12A-12C case 1 & 2, OUT2), which was done by adding the state substrate in large molar excess. Otherwise, for all other input probabilities (i.e., p_{b}_{₀}^{IN}(0)≠0.5) that may arise from the bit passing through a different gate (e.g., an L-gate), the U-gate outputs b₀ with the same probability distribution as the input (FIGS. 12A-12C, case 3). A single protease was computationally simulated (FIGS. 12D-12F) and experimentally validated (FIGS. 12G-12I) that two consecutive U-gate operations on b₀ (plasmin) in state 0 (cleaving GLQRALEI (SEQ ID NO:4)) would first output b₀ in state 0 and state 1 (cleaving KYLGRSYKV (SEQ ID NO:5)) with ˜50% probability each (45% and 55% respectively, n=3), and then reverse b₀ to exist in its original state 0 with ˜100% probability (93%, n=3).

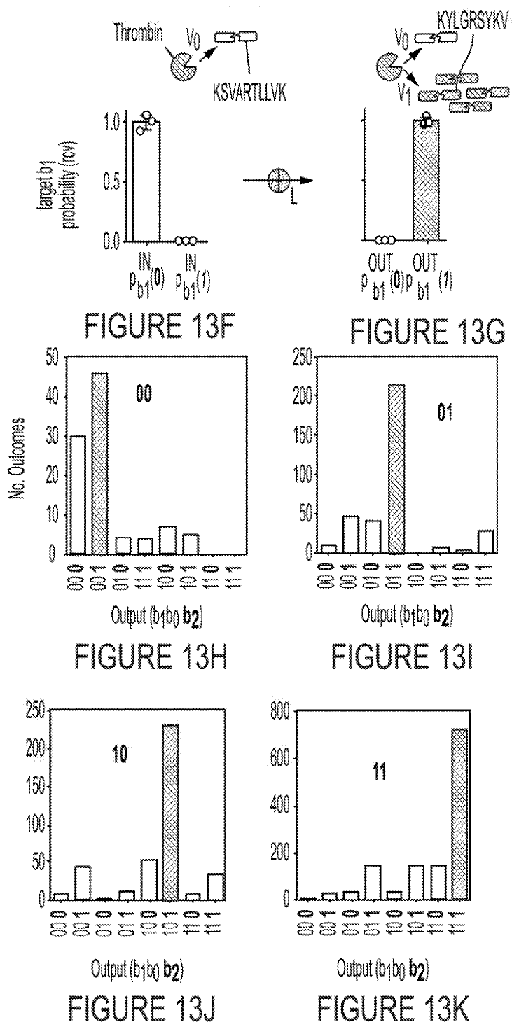

By contrast, analogous to a classical XOR gate, the L-gate was designed to link, or match, the state 1 probabilities of two bbits to the same value. The L-gate takes a control and target bbit (b₀ and b₁, respectively) and operates on the state 1 output probability of target b₁ (i.e., p_{b}_{₁}^{OUT}(1)=1−y where y=p_{b}_{₀}^{OUT}(0)) to match with the state 1 probability of control b₀ at a user-defined value, 1−y′ (i.e., make p_{b}_{₁}^{OUT}(1)=p_{b}_{₀}^{OUT}(1)=1−y′), that can take a probability between 0.5 and 1.0 to control the strength of the match between the bits (i.e., tune the likelihood that both bits would be found in state 1.0 simultaneously). This operation is applied if and only if the state 1 input probability of control b₀ is nonzero (i.e., p_{b}_{₀}^{IN}(1)≠0.0) (FIGS. 12J-12M, case 1); otherwise, the state 1 output probability of b1, p_{b}_{₁}^{OUT}(1) remains unchanged (FIGS. 12J-12M, case 2). For a matched output probability of 1−y′≈0.5, this was performed experimentally by adding substrate-1 (KYLGRSYKV (SEQ ID NO:5)) to control b₀ (e.g., plasmin) and target bi (e.g., thrombin) at a concentration such that the relative cleavage velocities (rcv) were matched (FIGS. 12N-12O; FIGS. 13A-13C). When using L-gates to solve the oracle, this was implemented by adding substrate-1 to control b₀ and target b₁ in large molar excess such that the relative cleavage velocities were at 1−y′≈1.0 (FIG. 13D-13E) so that the string bits occupying state-1 and the answer bit were fully matched (Table 3).

TABLE 3
Example Oracle Problem.

| bbit | Initial | | U-gate 1 | | U-gate 2 | | U-gate 3 | |
|---|---|---|---|---|---|---|---|---|
| (bₙ) | p(0) | p(1) | p(0) | p(1) | p(0) | p(1) | p(0) | p(1) |
| b₀ | 100 | 0 | 50 | 50 | 0 | 100 | 0 | 100 |
| b₁ | 100 | 0 | 50 | 50 | 50 | 50 | 100 | 0 |
| b₂ | 100 | 0 | 100 | 0 | 0 | 100 | 0 | 100 |

Using the U- and L-gates, biological analogs of probabilistic circuits called scores were constructed that were previously used to implement all four instances of the 2-bit LPN problem. Here U- and L-gates operate on three input protease bits, b₀-b₂, all initially in state 0 with 100% probability. These comprise 2 bits (b₀ and b₁) to represent possible hidden string values (00, 01, 10, and 11) and 1 answer bit (b₂) that is either linked to b₀, b₁, or b₀ and b₁ by a L-gate (for 01, 10, or 11 configurations respectively) or not at all (00 configuration). To solve the oracle scores, each U- or L-gate operation is applied in succession to generate final state 0 and 1 output probabilities for all three protease bits (Table 3). By multiplying all permutations of the output state 0 and 1 probabilities of bbits b₀-b₂ to estimate the joint probabilities (FIG. 11D), the protease solver correctly deduced the value of the hidden string among all other possibilities by assigning it the highest probability in all four oracle configurations (FIG. 12R-12S; Tables 4-5). Further, to validate these results computationally, the probabilistic Gillespie model was used to simulate three singular proteases, which solved all four oracle configurations with similar accuracy (FIGS. 13H-13K). Collectively, these results showed that protease activity can be quantified as state probabilities and operated by probabilistic logic gates to efficiently solve inference problems.

TABLE 4
Legend to identify proteases used as biological
bits to solve oracle problem.

| Oracle | | | |
|---|---|---|---|
| Configuration | b₀ (hidden string) | b₁ (hidden string) | b₂ (answer bit) |
| 00 | Granzyme B | C1r | Plasmin |
| 01 | Granzyme B | Thrombin | Plasmin |
| 10 | Thrombin | Granzyme B | Plasmin |
| 11 | Thrombin | Factor X1a | Plasmin |

TABLE 5
Legend to identify substrates for the proteases used in the oracle
problem.

| | | | SEQ |
|---|---|---|---|
| Protease Name | Abbreviation | Substrate(s) | ID NO: |
| Complement protease | C1r | 5FAM-QRQRIIGG-K(CPQ2)-C, | 17 |
| C1r | | 5FAM-LQRIYK-K(CPQ2)-C | 15 |
| Thrombin | Throm. | 5FAM-KSVARTLLVK-K(CPQ2)-C, | 16 |
| | | 5FAM-KYLGRSYKV-K(CPQ2)-C | 18 |
| Plasmin | Plasm. | 5FAM-GLQRALEI-K(CPQ2)-k-C, | 19 |
| | | 5FAM-KYLGRSYKV-K(CPQ2)-C | 18 |
| Factor XIa | Fac. XIa | 5FAM-LQRIYK-K(CPQ2)-C, | 15 |
| | | 5FAM-KYLGRSYKV-K(CPQ2)-C | 18 |
| Granzyme B | GzmB | 5FAM-aIEFDSGK(CPQ2)kkc | 7 |

### Example 5. Sampling Multi-State Probabilistic Protease Bits to Detect Thrombosis

### Materials and Methods

Nanosensor synthesis and characterization: Aminated IONPs were synthesized in house per published protocol 27. Mass barcode-labelled substrate peptides synthesized by MIT Core Facility and used for in vivo formulation. Aminated IONPs were first reacted to the heterobifunctional crosslinker Succinimidyl Iodoacetate (SIA; Thermo) for 2 hours at room temperature (RT) and excess SIA were removed by buffer exchange using Amicon spin filter (30 kDa, Millipore). Sulfhydryl-terminated peptides and Polyethylene Glycol (PEG; LaysanBio, M-SH-20K) were mixed with NP-SIA (90:20:1 molar ratio) and reacted overnight at RT in the dark to obtain fully conjugated activity nanosensors. Activity nanosensors were purified on a Superdex 200 Increase 10-300 GL column using AKTA Pure FPLC System (GE Health Care). Ratios of FITC per IONP were determined using absorbance of FITC (488 nm, ε=78,000 cm−1M−1) and IONP (400 nm, ε=2.07×106 cm−1M−1) (Titball, R. W., Microbiol Rev, 57:347-366 (1993) measured with Cytation 5 Plate Reader (Biotek). At this conjugation condition, the resulting formulations have an average of 50 FITC-labelled peptides per nanoparticle core. DLS measurements of activity nanosensors were done in PBS or mouse plasma at RT using Zetasizer Nano ZS (Malvern).

Urinary Prediction of Blood Clots in Murine Model of Pulmonary Embolism: All urinalysis experiments were done in paired setup. Before (4 days prior) onset of thrombosis, mice were administered with peptide substrate-labelled activity nanosensors (50 μg of IONP per animal). To collect urine, mice were placed over 96-well polystyrene plates surrounded by an open cylindrical sleeve covered by a weighted petri dish to prevent animals from leaving the cylinder. Thrombosis, or pulmonary embolism, was initiated by coinjecting 1.75 μg /g b.w. of rabbit tissue factor and 0.17 mg/mouse of VT750-labeled fibrinogen (0.5 nmol). VT750-fibrinogen was co-infused to allow detect of newly formed fibrin clots by fluorescent near-infrared imaging of excised organs. Control mice received fibrinogen-VT750 alone to measure background fluorescence. Starting immediately after tissue factor infusion, animals were left to urinate for 30 minutes before urine samples were collected. Individual substrates were quantified by mass spectrometry, which was performed as a service by Syneos Health. Morbidity to treatment included shortness of breath, decrease in activity, and slightly raised fur. Mortality rate for all experiments was 2/27 (7%).

Description of computational model for simulating the statistical sampling of protease bits: To model networks of protease activity for a human patient, baseline activity scores between zero and one were randomly generated for all 550 proteases encoded in the human genome (i.e., ground truth string for a healthy patient) (Lopez-Otin & Bond, J Biol Chem, 283:30433-30437 (2008)). These activity scores represent the fraction of the maximum activity for each protease in a healthy state. To simulate protease dysregulation that occurs in human disease, random subsets of 0, 20, 100, or 550 proteases were chosen to be upregulated or downregulated by scaling their activity by a factor of five to reflect an average of literature reported values based on RNA fold-change (Krochmal, et al., Scientific Reports, 7:15160 (2017); Tarca, et al., Am J Obstet Gynecol, 195(2):373-388 (2006); Kappelhoff, et al., Biochimica et Biophysica Acta, 1864:2210-2219 (2017)). To act as a positive control, all 550 proteases in the disease networks were dysregulated, and as a negative control, the activity values of disease-related protease activity were not scaled. To simulate promiscuous sampling, a substrate library of size m (ranging from 2-550) was modeled randomly sampling n proteases (ranging from 1-550) by adding corresponding activity scores. From these substrate values, the associated probability distribution and variance across all substrates were computed and these values were used as a binary classifier to separate healthy networks of protease activity from disease networks. To examine the effect of the size of the peptide substrate library on classification accuracy (i.e., AUROC), this protocol was iterated while increasing the number of substrates used to sample the protease bits.

### Results

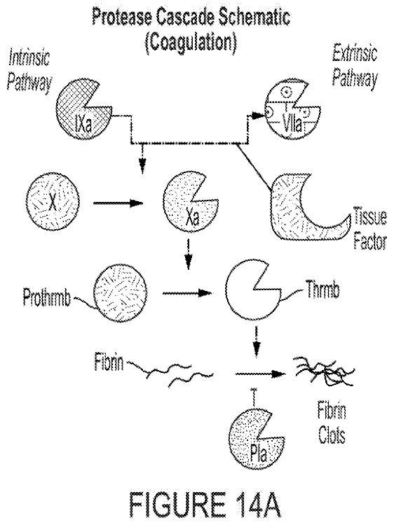

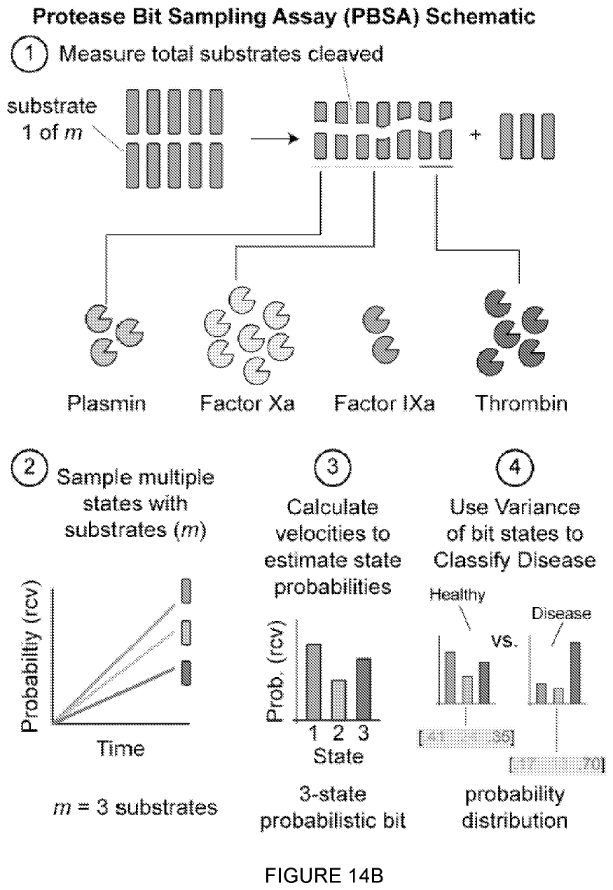

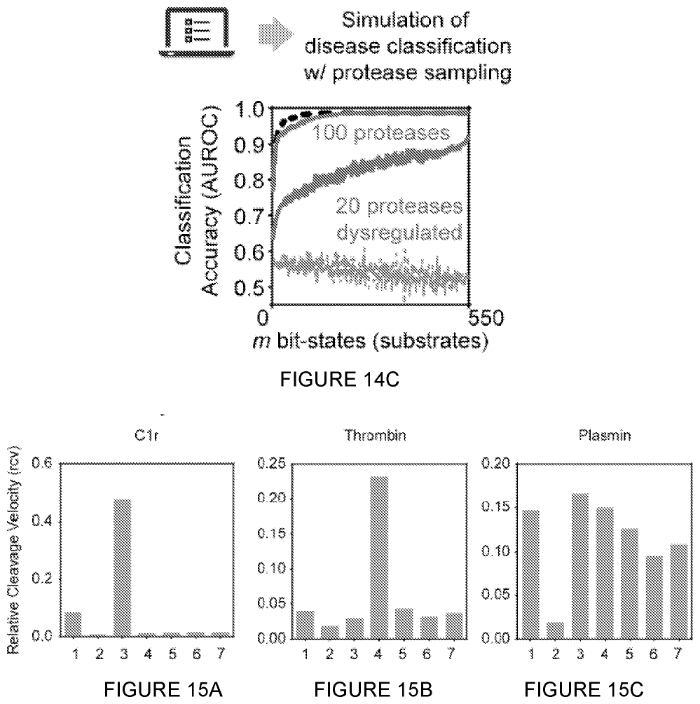

The dysregulated activity of protease networks is a hallmark of complex diseases, which has provided the impetus to use measurements of protease activity as biomarkers (Lopez-Otin and Bond, J Biol Chem, 283:30433-30437 (2008); Holt, et al., JoVE, e57937 (2018); Dudani, et al., Adv Funct Mater, 26(17):2919-2928 (2016); Fonovic & Bogyo, Curr Pharm Des, 13(3):253-261 (2007); Mac, et al., Nat Biomed Eng, 3:281-291 (2019)). Therefore, the concept of protease bits was extended to biomedical diagnostics by considering the activity of a network of dysregulated proteases (e.g., the coagulation cascade, FIG. 14A) cleaving a set of m promiscuous substrates as a m-state probabilistic bit (FIG. 14B). Identical to two-state probabilistic bits, the relative cleavage velocities (rcv) of the m-substrates represent a m-state probability distribution. It was postulated that by using the same set of promiscuous m-substrates to sample different protease networks (e.g., coagulation vs. complement cascade), each network would result in distinct m-state probability distributions (FIG. 14B) that classify disease and healthy samples (e.g., by the variance of the probability distributions). This probability-based method is called the protease bit sampling assay (PBSA).

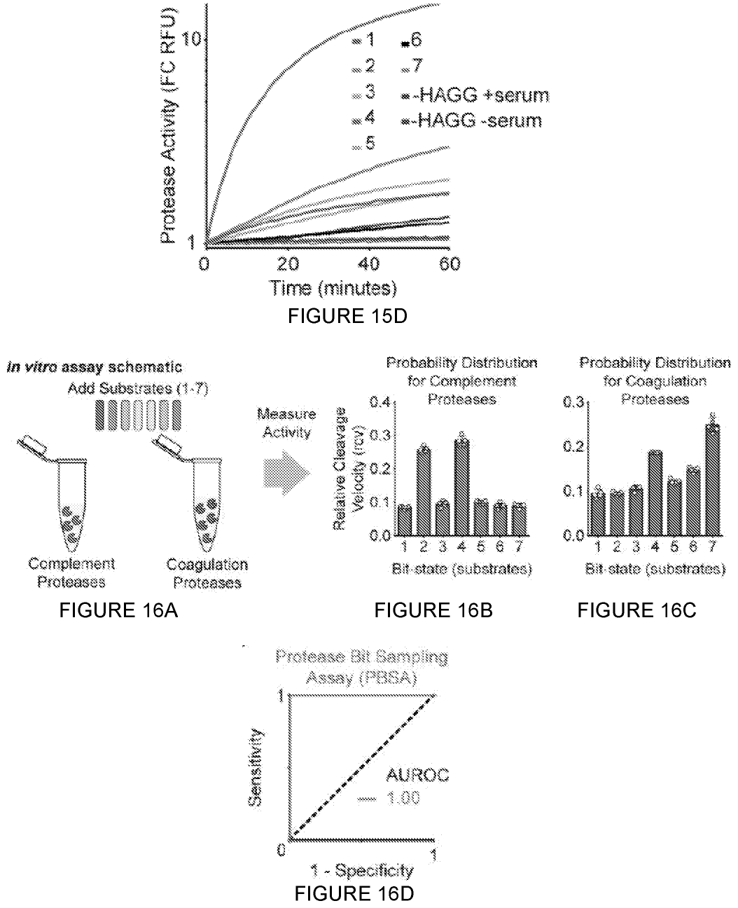

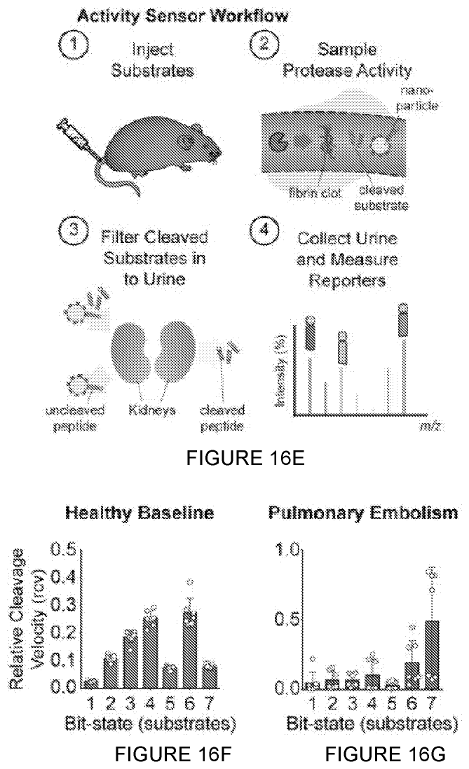

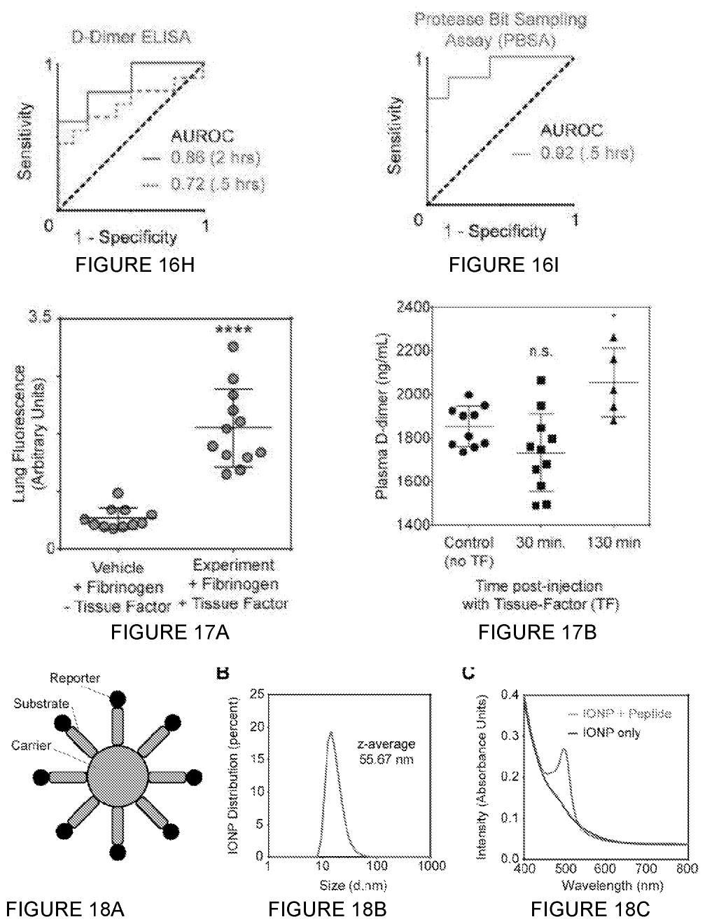

To test this approach computationally, a simulation was developed to quantify the ability to classify diseased from healthy protease networks by PBSA. The results from the model revealed that the ability to classify disease and healthy networks increases as the number of dysregulated proteases (red and green trace) or m-state substrates increases (e.g., greater than 90% classification accuracy can be achieved with >10 m-state substrates and >20 dysregulated proteases) (FIG. 14C). This result showing dependence of classification accuracy on feature size was consistent with computational results based on multidimensional datasets (Zhao, et al., Briefings in Bioinformatics, 16:291-303 (2015)). To validate the model prediction, common-target substrates (numbered 1-7) were designed as a 7-state bbit system to promiscuously sense the complement (e.g., C1r, MASP2, Factor D, Factor I) and coagulation protease networks (e.g., thrombin, plasmin, factor XIIa, factor Xa, protein C) (FIGS. 15A-15D). By quantifying relative cleavage velocities (rcv) as 7-state probability distributions after incubation with either group of proteases in vitro (FIG. 16A), the normalized variances of the resulting probability distributions classified mixtures as either complement or coagulation with perfect accuracy (n=10, AUROC=1.00, FIGS. 16B-16D). These results confirmed that a set of promiscuous substrates can be used to sample and discriminate differences in the underlying probability distributions of protease bits in enzymatic networks.

To apply PBSA for in vivo diagnostics, an established mouse model of thrombosis where tissue factor (TF) is intravenously administered to induce the formation of blood clots that then embolize to the lung was used (Weiss, et al., Blood, 100:3240-3244 (2002); Leon, et al., Circulation, 103:718-723 (2001); Smyth, et al., Blood, 98:1055-1062 (2001)). To validate the rate of disease penetrance, mice (n=12) were co-infused with tissue factor and fibrinogen labeled with a near-infrared dye (VT750) to allow detection of newly formed fibrin clots by near-infrared fluorescent imaging of excised lungs. Compared to control mice (n=11) infused with VT750-fibrinogen alone, a significant increase in overall lung fluorescence was observed in all TF-infused mice (FIG. 17A) but no differences in fluorescence was observed in major organs (e.g., heart, spleen, lymph nodes, kidney, liver, lungs). The induction of thrombosis was further validated by quantifying plasma levels of d-dimer, a clinically used biomarker that is released as a byproduct of fibrinolysis. Significant elevations in d-dimer levels were detected ˜130 minutes after TF infusion, but not at ˜30 minutes, which was attributed to an early timepoint before onset of fibrinolysis (FIG. 17B).

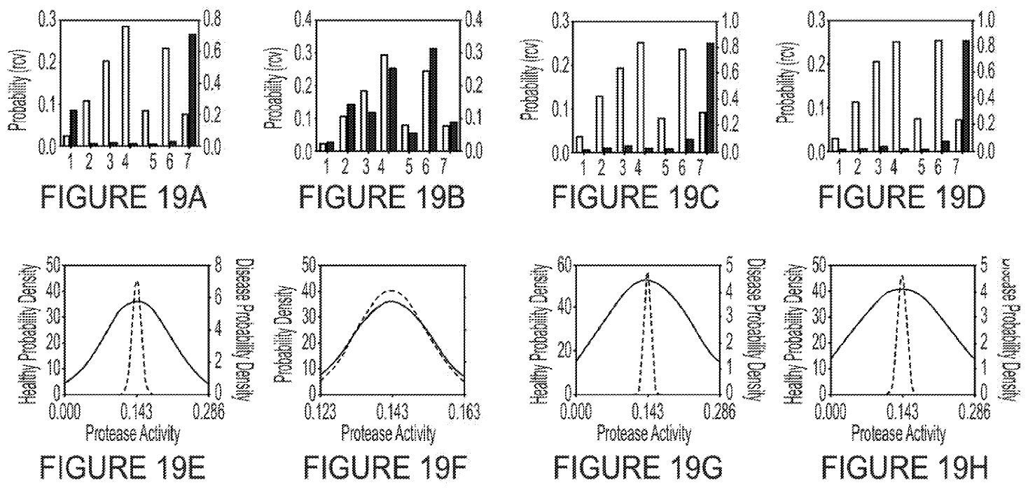

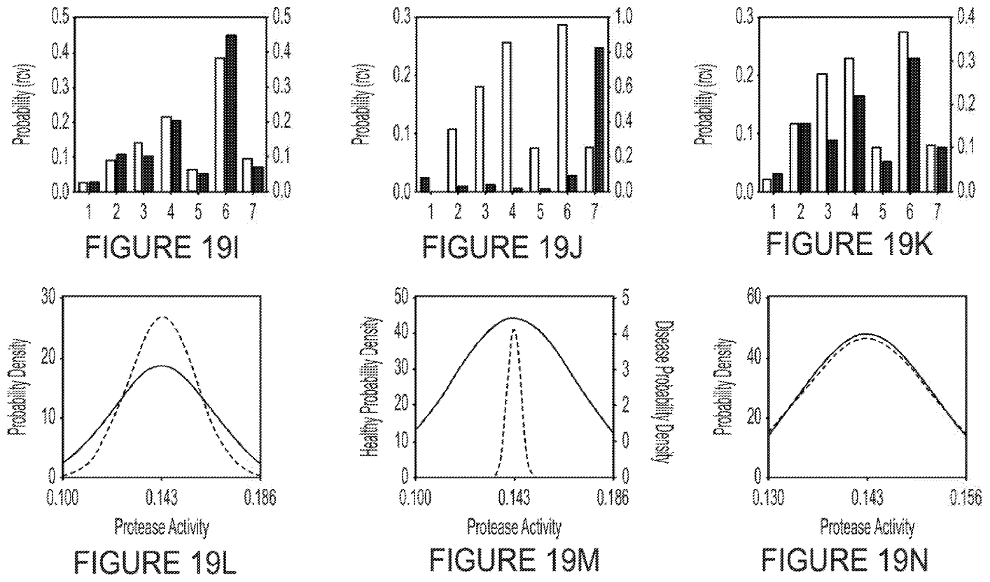

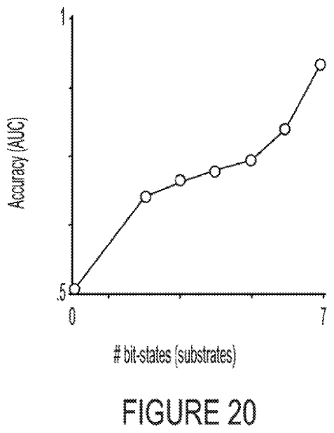

To apply PBSA to discriminate thrombosis from healthy controls in mice, a class of mass-barcoded protease activity sensors that were previously developed (Holt, et al., JoVE, e57937 (2018); Kwong, et al., PNAS, 112:12627-12632 (2015); Kwong, et al., Nat Biotech, 31:67-70 (2013)) were used for multiplexed quantification of protease activity in vivo. These probes included state substrates 1-7 each uniquely labeled with a mass-barcode (i.e., reporter), which are then conjugated to iron oxide nanoparticles (IONPs) (i.e., carrier) (FIGS. 18A-18C). After intravenous infusion, circulating substrates on the IONPs are cleaved by coagulation proteases, releasing peptide fragments that clear into urine. The urine samples are then collected, and the peptide fragments quantified by tandem mass spectrometry according to their mass barcode (FIG. 16E). Using this platform, a single cocktail of the seven mass-barcoded state substrates were administered in healthy mice (Holt, et al., JoVE, e57937 (2018)) and the cleaved peptide levels were quantified in urine (t=30 min) to establish a healthy baseline of activity (FIG. 16F-16G). After 4 days to allow full clearance of the sensors (Kwong, et al., Nat Biotech, 31:63-70 (2013); Mac, et al., Nat Biomed Eng, 3:281-291 (2019)), the same cohort of animals was infused with TF to induce thrombosis, which was immediately followed by a second administration of mass-barcoded state substrates, collection of urine samples (t=30 min), and quantification of cleaved peptide substrates in urine by mass spectrometry (FIG. 16F-16G). The cleaved fragments of substrates 1-7 in urine were then quantified as relative cleavage probabilities. By using the calculated sample variances across the 7-state probability distribution, PBSA discriminated TF-induced thrombosis mice from healthy controls with high sensitivity and specificity (AUROC=0.92). By comparison, d-dimer classification resulted in AUROC of 0.72 and 0.86 at 30 and 130 minutes post TF infusion respectively (FIG. 16H-16I FIGS. 19A-19N). Consistent with the mathematical predictions, overall classification accuracy increased from 0.5 to 0.92 as the number of bit-states (i.e., substrates) used in the classifier increased from zero to seven, respectively (FIG. 20). Collectively, the data showed that by treating networks of protease activity as a m-state probabilistic bit, the underlying variance in states can be used to infer and diagnose disease with high accuracy.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

1. A drug delivery circuit comprising:
a plurality of biocomparators, and
a plurality of activatable drug delivery agents, wherein the plurality of biocomparators perform a round of computation to determine if conditions are present to activate the drug, and if so then activate the appropriate drug delivery agent to deliver a drug to a subject.

2. The drug delivery circuit of claim 1, wherein the biocomparators comprise a biologically inert structure having a core and being surrounded by a density of cleavable peptides.

3. The drug delivery circuit of claim 1, wherein the activatable drug delivery agents are activated by cleavage of a peptide surrounding the agent.

4. The drug delivery circuit of claim 3, wherein the agent is cleaved by a protease or a chemical.

5. The drug delivery circuit of claim 1, wherein the biocomparator core comprises agents to activate the drug delivery agent.

6. The drug delivery circuit of claim 1, wherein the biocomparator core comprises proteases and protease inhibitors, wherein the proteases activate the drug delivery agents and the inhibitors inhibit the proteases such that the unique combination of protease and inhibitor cleaves only a specific drug delivery agent.

7. The drug delivery circuit of claim 1, wherein the biocomparators sense the amount of diseased cells or infectious agent in a sample or subject.

8. The drug delivery circuit of claim 1, wherein the amount of diseased cells or infectious agent in a sample or subject cause one of the plurality of biocomparators to open and release the contents of the core, wherein the contents of the core cleave a specific drug delivery agent.

9. The drug delivery circuit of claim 1, wherein the plurality of activatable drug delivery agents comprise different doses of a therapeutic agent.

10. A method for treating disease in a subject in need thereof, comprising:
contacting a biological sample from the subject with a plurality of biocomparators, wherein each biocomparator is surrounded by peptides that can be cleaved, each of the plurality of biocomparators comprises a different density of peptide surrounding it, and each of the plurality of biocomparators comprises a unique detectable molecule within its core;
detecting the molecules released from each of the plurality of biocomparators;
assigning a binary digit to the sample, wherein each detectable molecule is pre-assigned a binary digit that corresponds to a threshold protease level, and the presence of each detectable molecule or combination of detectable molecules present in the sample determines the binary digit;
diagnosing the subject with disease if the binary digit indicates the protease level is above the threshold level; and
administering to the subject the minimum effective dose of a therapeutic agent necessary to treat the disease or disorder.

11. The method of claim 10, wherein the detectable molecule within the biocomparator comprises a fluorescent molecule, a bioluminescent molecule, or a mass-tag.

12. The method of claim 10, wherein the detectable molecule within the biocomparator comprises a protease substrate flanked by a quencher molecule and a fluorescent molecule.

13. The method of claim 10, wherein the biocomparator further comprises signal proteases and protease inhibitors.

14. The method of claim 10, wherein the sample comprises a urine sample or a blood sample.

15. The method of claim 10, wherein detecting the molecules comprises subjecting the sample to mass spectrometry, flow cytometry, or ELISA.

16. The method of claim 10, wherein the subject has cancer.

17. The method of claim 10, wherein the subject has a bacterial or viral infection.

18. The method of claim 10, wherein the therapeutic agent is an immunotherapeutic or immunosuppressive agent.

19. The method of claim 10, wherein the therapeutic agent is an antimicrobial agent.

20. The method of claim 10, wherein the peptides can be cleaved by a cancer-associated protease such as but not limited to matrix metalloproteinases, kallikreins, ADAM10, ADAM 17, cathepsin B, cathepsin L, cathepsin S, uPA, uPAR, PSA, and caspases-3, -6, -7, and -8.

21. The method of claim 10, wherein the peptides can be cleaved by bacterial peptides such as OmpT.

22.-36. (canceled)
